(19) United States
(12) Patent Application Publication
(10) Pub. No.: US 2023/0312433 A1
(43) Pub. Date: Oct. 5, 2023

(54) SELECTIVE ISOBUTANE OXIDATION TO TERT-BUTANOL IN THE PRESENCE OF CUBANE CLUSTERS CATALYST

(71) Applicant: ExxonMobil Technology and Engineering Company, Annandale, NJ (US)

(72) Inventors: Jihad M. Dakka, Whitehouse Station, NJ (US); David O. Marler, Easton, PA (US); Avelini Corma Canos, Valencia (ES); Pasquel Ona Burgos, Valencia (ES)

(21) Appl. No.: 17/650,840

(22) Filed: Feb. 11, 2022

(51) Int. Cl. C07C 1/22; C07C 29/48; C07C 45/27; B01J 31/18; C07C 5/22; B01J 23/52; B01J 23/10; B01J 35/02; B01J 23/46; B01J 21/06

(52) U.S. Cl. CPC C07C 1/22; B01J 21/063; B01J 23/10; B01J 23/462; B01J 23/52; B01J 31/1815; B01J 35/026; C07C 5/22; C07C 29/48; C07C 45/27; B01J 2231/70

(57) ABSTRACT

The present disclosure provides catalyst compositions and processes for the conversion of low-cost short chain alkanes to high value liquid transportation fuels and chemicals. The present disclosure provides methods of making said catalyst compositions.

### FIELD

The present disclosure provides catalyst compositions and processes for the conversion of low-cost short chain alkanes to high value liquid transportation fuels and chemicals. The present disclosure provides methods of making said catalyst compositions.

### BACKGROUND

As the production of shale and tight oils is increasing in the United States of America, light paraffins (e.g., C₃ to C₉), such as Liquefied Petroleum Gas (“LPG”), Natural Gas Liquids (“NGL”), are becoming increasingly abundant and at lower costs. Ethane to light naphtha range paraffins are largely fed to steam crackers or dehydrogenated to make olefins. For example, ethane is steam-cracked to make ethylene, and light naphtha (b.p. 15.5° C. - 71° C.) is steam cracked to make ethylene, propylene, and small volumes of dienes.

Short-chain alkanes (e.g., C₂-alkanes to C₅-alkanes) can also be converted to their corresponding olefin using dehydrogenation technologies. Dehydrogenation of short-chain alkanes (e.g., C₂ to C₅) commonly uses one of two types of catalysts: platinum-based catalyst(s) or chromium oxide catalyst(s). The dehydrogenation process is typically carried out at temperatures > 450° C., and under ambient or sub-ambient pressure, mainly due to the fact that paraffin dehydrogenation to olefins, or dehydrogenative coupling to heavier paraffins, are both thermodynamically unfavored and conversion is equilibrium limited. Hence, the free energy of the dehydrogenation reaction only becomes favorable at temperatures of at least 600° C. To manage the frequency of a catalyst regeneration process due to coking, reactors such as moving-bed, cyclic swing-bed, or fluidized bed reactors are employed.

Conversion of light paraffins to distillate is typically performed using the following technologies: 1) steam cracking or catalytic dehydrogenation of paraffins to generate olefins, followed by olefin oligomerization; 2) converting the feed to syngas via partial oxidation, followed by Fischer-Tropsch or methanol to hydrocarbons synthesis. However, these approaches involve high temperatures (e.g. >400° C.) and are energy intensive.

Because of increasing demand for higher octane and lower reid vapor pressure (RVP) gasoline, increasing supply of light paraffins associated with shale gas, high cost of olefins and safety issues with the use of HF and H₂SO₄, there is a desire for a technology which offers solutions to all of these issues. Also there is a desire for a technology that will offer the production of chemicals building blocks e.g., propylene, form non-conventional feed and technologies.

For example, although commercial processes exist that convert isobutane to ter-butyl hydrogen peroxide (TBHP) and tert-butyl alcohol (TBA), the objective of the commercial process is to maximize the selectivity to TBHP and minimize the selectivity to TBA. In order to do that, these processes run at low temperature and long residence time to prevent the TBHP decomposition to TBA.

As such, there remains a need for processes that provide a highly efficient and highly selective oxidation and conversion of low-cost short chain alkanes to high value liquid transportation fuels and chemicals at faster reaction rates and higher selectivity via efficiently coupled catalytic process steps.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

This invention provides for a method of oxidizing a linear or branched alkane to produce an oxygenate species, including but not limited to, and alcohol species and a method for the production of distillate range products, including but not limited to high-octane fuel and gasoline products, from the alkanes.

In one aspect, the invention provides for a method for the production of distillate range products from light alkanes comprising:
(A) a step of oxidixing a linear or branched alkane to produce an oxygenate species;
(B) a step of condensing the oxygenate species to produce a condensed species;
and (C) a step of hydro-finishing the condensed species to produce distillate range products,
wherein the step of oxidizing a linear or branched alkane comprises reacting the alkane in the presence of one or more catalysts, wherein at least one catalyst is a cobalt cubane cluster catalyst.

In certain embodiments of the method of invention, the alkane is a light alkane; including, but not limited to n-butane.

In some embodiments of the method of the invention, the cobalt cubane cluster catalyst has the formula:

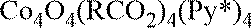

wherein R represents an aromatic or aliphatic group; and
Py* is pyridine or a pyridine functionalized at 4 position.

In particular embodiments of the method of the invention, wherein R is methyl or phenyl. In other particular embodiments, Py* is pyridine, 4-methoxy-pyridine, or 4-ethoxycarbonyl-pyridine.

In certain embodiments of the method of the invention, the cobalt cubane cluster catalyst has the formula:

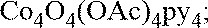

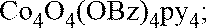

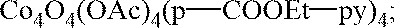

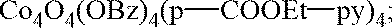

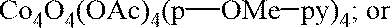

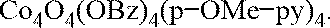

In some embodiments of the method of the invention, the cobalt cubane cluster catalyst is present in an amount of about 0.001 ppm to about 10 ppm of the total moles of reactants.

In other embodiments of the method of the invention, the step of oxidizing a linear or branched alkane is performed in the presence of a cobalt cubane cluster catalyst and an additional catalyst. In certain embodiments, the additional catalyst is a catalyst comprising palladium, ruthenium, magnesium, titanium, cerium, vanadium, manganese, nickel, zinc, tin, cobalt, silver, gold, platinum, or lanthanum or mixtures thereof. In particular embodiments, the additional catalyst is La₂O₃, CuO, MgO, CeO₂, TiO₂, V₂O₃, CoOₓ, MnOₓ, Au/CeO₂, Ru/CeO₂, or Ru/TiO₂ catalyst. In still other embodiments, the additional catalyst is present as a nanoparticle.

In certain embodiments of the method of the invention, the additional catalyst is present in an amount of about 0.001 mol % to about 5 mol % of the total moles of reactants. In still other embodiments of the method of the invention, the additional catalysts are present in an molar ratio of 1,000:1 to 1:1,000 with respect to the number of moles of cobalt cubane cluster.

In some embodiments of the method of the invention, the step of oxidizing a linear or branched alkane is performed in the presence of a solvent. IN particular embodiments, the solvent includes benzonitrile in an amount from 10 wt % to 90 wt % of the total solvent.

In other embodiments of the method of the invention, the step of oxidizing a linear or branched alkane is performed under supercritical conditions.

In still other embodiments of the method of the invention, the method further comprises a step of isomerizing a linear alkane to form a branched alkane.

In still yet other embodiments of the method of the invention, the step of oxidizing a linear or branched alkane selectively forms the alcohol component in an amount of greater than 60%.

These and other features and attributes of the disclosed catalyst compositions and processes of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings, wherein:”

FIG. 1 depicts the process scheme for the conversion of n-butane to ethylene propylene, propylene, isooctane, and jet fuel/diese.

FIG. 2 depicts the mechanism associated to the clusters {Co₄O₄} in the O—O bond formation.

FIG. 3 is a three-dimentional illustration of the cubane clusters of complexes 3, 4, and 6 described in the examples.

FIG. 4a is the ¹H -NMR of [Co₄O₄](OAc)4(py-CO2Et)4 3 in DMSO-d6.

FIG. 4b is the ¹H -NMR of [Co4O4](PhCO2)4(py-CO2Et)4 4 in DMSO-d6.

FIG. 5 is a graph depicting the oxidation potential for each cobalt cluster respect to their sigma parameter in Hammet equation.

FIG. 6. is an illustration of the of the CV curve of clusters of complexes 1-6 described in the examples.

FIG. 7 is the raman spectrum of complex 3 described in the examples.

FIG. 8a is a graph depicting the conversion of isobutane reaction at different temperatures for Au/ CeO₂, cubane 4, and a cixture of Au/ CeO₂, and cubane 4.

FIG. 8b is a graph depicting the selectivity of the isobutane reaction at different temperatures for Au/ CeO₂, cubane 4, and a cixture of Au/ CeO₂, and cubane 4.

FIG. 9 is a plot depicting catalyst screening and conditions for isobutene oxidation.

FIG. 10 is a plot depicting selectivity versus conversion at 2 hours of reaction time employing La₂O₃ catalysts. Green circles 130° C.; Blue circles supercritical conditions.

FIG. 11 is a plot depicting selectivity versus conversion at 4 hours of reaction time employing La₂O₃ catalysts. Green circles 130° C.; Blue circles supercritical conditions.

### DETAILED DESCRIPTION

Throughout the entire specification, including the claims, the following terms shall have the indicated meanings. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

A/an: The articles “a” and “an” as used herein mean one or more when applied to any feature in embodiments and implementations of this disclosure described in the specification and claims. The use of “a” and “an” does not limit the meaning to a single feature unless such a limit is specifically stated. The term “a” or “an” entity refers to one or more of that entity. As such, the terms “a” (or “an”), “one or more” and “at least one” can be used interchangeably herein.

About: As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly,” consistent with the discussion below regarding ranges and numerical data. All numerical values within the detailed description and the claims herein are modified by “about” or “approximately” the indicated value, and take into account experimental error and variations that would be expected by a person having ordinary skill in the art.

And/or: The term “and/or” placed between a first entity and a second entity means one of (1) the first entity, (2) the second entity, and (3) the first entity and the second entity. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements). As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”.

Comprising: In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. Any device or method or system described herein can be comprised of, can consist of, or can consist essentially of any one or more of the described elements.

Ranges: Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and about 200, but also to include individual sizes such as 2, 3, 4, etc. and sub-ranges such as 10 to 50, 20 to 100, etc. Similarly, it should be understood that when numerical ranges are provided, such ranges are to be construed as providing literal support for claim limitations that only recite the lower value of the range as well as claims limitation that only recite the upper value of the range. For example, a disclosed numerical range of 10 to 100 provides literal support for a claim reciting “greater than 10” (with no upper bounds) and a claim reciting “less than 100” (with no lower bounds). In the figures, like numerals denote like, or similar, structures and/or features; and each of the illustrated structures and/or features may not be discussed in detail herein with reference to the figures. Similarly, each structure and/or feature may not be explicitly labeled in the figures; and any structure and/or feature that is discussed herein with reference to the figures may be utilized with any other structure and/or feature without departing from the scope of the present disclosure.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

The term “active” refers to substance having an element or compound that participates as a reactant in a chemical reaction and may optionally have catalytic characteristics.

The term “alkane” means substantially saturated compounds containing hydrogen and carbon only, e.g., those containing ≤1% (molar basis) of unsaturated carbon atoms. The term alkane encompasses C₂ to C₆ linear, iso, and cyclo alkanes.

The term “Cₙ” hydrocarbon wherein n is a positive integer, e.g., 1, 2, 3, 4, or 5, means hydrocarbon having n carbon atom(s) per molecule.

The term “Cₙ₊” hydrocarbon wherein n is a positive integer, e.g., 1, 2, 3, 4, or 5, means hydrocarbon having at least n carbon atom(s) per molecule.

The term “Cₙ₋” hydrocarbon wherein n is a positive integer, e.g., 1, 2, 3, 4, or 5, means hydrocarbon having no more than n number of carbon atom(s) per molecule.

The term “cycle time” means the time from a first interval to the next first interval, including (i) intervening second, third, and/or fourth intervals and (ii) any dead-time between any pair of intervals.

The term “flow-through reactor” refers to a reactor design in which one or more reagents enter a reactor, typically an elongated channel or stirred vessel, at an inlet, flow through the reactor, and then a product mixture (including any unreacted reagents) is continuously or semi-continuously collected at an outlet. Flow-through reactors include continuous reactors, as well as semi-continuous reactors in which one phase flows continuously through a vessel containing a batch of another phase, e.g., fixed-bed reactors where a fluid phase passes through a solid phase of catalyst, reactant, active material, etc.

With respect to flow-through reactors, the term “region” means a location within the reactor, e.g., a specific volume within the reactor and/or a specific volume between a flow-through reactor and a second reactor, such as a second flow-through reactor. With respect to flow-through reactors, the term “zone”, refers to a specific function being carried out at a location within the flow-through reactor. For example, a “reaction zone” or “reactor zone” is a volume within the reactor for conducting at least one of oxidative coupling, oxydehydrogenation and dehydrocyclization. Similarly, a “quench zone” or “quenching zone” is a location within the reactor for transferring heat from products of the catalytic hydrocarbon conversion, such as C₂₊ olefin.

The term “hydrocarbon” means compounds containing hydrogen bound to carbon, and encompasses (i) saturated hydrocarbon, (ii) unsaturated hydrocarbon, and (iii) mixtures of hydrocarbons, including mixtures of hydrocarbons (saturated and/or unsaturated) having different values of n.

The term “oxidant” means any oxygen-bearing material which, under the conditions in the reaction zone, yields oxygen for transfer to the oxygen storage material, for storage with and subsequent release from the oxygen storage material to the oxidative coupling and/or oxydehydrogenation. While not wishing to be limited to theory, molecular oxygen atoms may be provided as a reactive gas in a gaseous zone and/or atomic oxygen may be provided from a catalyst surface as, for instance, reacted, sorbed forms.

The terms “oxidized state” and “reduced state” refer to relative states of oxidation and reduction with respect to a reference state. For example, in compositions of the formulae Mn⁺²_{A1}Mn⁺³_{B1}Oₓ and Mn⁺²_{A2}Mn⁺³_{B2}O_{y}, where x<y, A1>A2, and B1<B2, Mn⁺²_{A1}Mn⁺³_{B1}Oₓ is the reduced state compound and Mn⁺²_{A2}Mn⁺³_{B2}O_{y} is the oxidized state compound.

The term “oxydehydrogenation” means oxygen-assisted dehydrogenation of an alkane, particularly a C₂+ alkane, to produce an equivalent alkene and water.

The term “reaction stage” or “reactor stage” means at least one flow-through reactor, optionally including means for conducting one or more feeds thereto and/or one or more products away therefrom.

The term “residence time” means the average time duration for non-reacting (non-converting by oxidative coupling) molecules (such as He, N₂, Ar) having a molecular weight in the range of 4 to 40 to traverse the reactor or a defined zone within the reactor, such as a reaction zone of a oxidative coupling reactor.

The term “spinel” refers to the cubic crystalline structure of the spinel class of minerals typified by the mineral spinel, MgAl₂O₄, or a material having such a structure. A spinel has the general formula AB₂X₄, where X is an anion such as chalcogen, e.g., oxygen or sulfur, arranged in a cubic close-packed lattice, and A and B are cations, which may be different or the same, occupying some or all of the octahedral and tetrahedral sites in the lattice, also including the so-called inverse spinels where the B cations may occupy some or all of the typical A cation sites and vice versa. Although the charges of A and B in the prototypical spinel structure are +2 and +3, respectively, i.e., A²⁺B³⁺₂X²⁻₄, other combinations incorporating divalent, trivalent, or tetravalent cations, including manganese, aluminum, magnesium, zinc, iron, chromium, titanium, silicon, and so on, are also possible.

The term “unsaturated” means a Cₙ hydrocarbon containing at least one carbon atom directly bound to another carbon atom by a double or triple bond.

As used herein, and unless otherwise indicated, a “metal oxide” refers to a metal oxide reagent/reactant that is reduced during a dehydrogenation process of the present disclosure. In comparison, a metal oxide catalyst would be regenerated to its original form (e.g. oxidation state) during a chemical reaction. Metal oxide reagents/reactants of the present disclosure can be regenerated from their reduced forms by treating the reduced form of the metal oxide to an oxidizing agent, as described in more detail below.

Dehydrogenation can reduce the first metal oxide to form a second metal oxide, also referred to as “a reduced metal oxide”. Methods may include: i) introducing the reduced metal oxide to a catalytic oxidation unit; ii) and regenerating the first metal oxide in the catalytic oxidation unit by contacting the second metal oxide with an oxidizing agent (e.g., air).

### Process for Conversion

This application relates to production of distillate range products from light alkanes (i.e., short chain (C1 to C8) alkanes), and, more particularly, to embodiments related to a methods and systems to produce distillate range products from oxygenate intermediates. While the methods and systems disclosed herein may be suitable to provide distillate range products in a standalone unit, the methods and systems may be particularly suitable for an integrated process within a petroleum refinery or chemical processing plant.

There may be several potential advantages to the methods and systems disclosed herein, only some of which may be alluded to in the present disclosure. One of the many potential advantages of the methods and systems is that the inefficiencies from utilizing on-purpose olefin production for production of distillate range products may be addressed. As discussed above, steam cracking and dehydrogenation may be two processes which produce on-purpose olefins. Catalytic alkane dehydrogenation to produce olefins typically requires high temperatures, low pressure, and frequent catalyst regeneration. Dehydrogenation methods may be limited by low equilibrium conversions due to the endothermic nature of the dehydrogenation reactions. The relatively low-per pass conversion in dehydrogenation methods may lead to a large recycle ratio. Steam cracking of naphtha to produce olefins also requires high temperatures, low naphtha partial pressure, and the reactor may be readily fouled by coking reactions. In either process, process conditions which favor olefin production are high temperature (e.g., >840° F. or 450° C.) and low pressure (ambient or vacuum). These process conditions are often satisfied by supplying large amounts of heat to the reactor to overcome the equilibrium constraint to reach appreciable per-pass olefin conversion. Products of dehydrogenation and steam cracking often require cryogenic separation and compression which adds to the energy requirement of the naphtha to olefins conversion process. In a typical steam cracker, olefins production accounts for approximately one-third of the overall unit operational cost, and olefins separation accounts for approximately two-thirds of the overall unit operational cost. After the olefins are produced either by steam cracking or dehydrogenation, the olefins may be oligomerized to distillate range products. The carbon number distribution of the products may depend on feed composition, catalyst, and process conditions. The distillate range products produced from olefin intermediates may be expensive due to the large energy requirement of olefin production and separation.

Embodiments may include an integrated process for production of distillate range products from naphtha range alkanes via oxygenate intermediates

Distillate range products may be an industry term used to identify a cut of hydrocarbon products produced in a refinery. Distillate range products may be produced in various units in a refinery such as atmospheric distillation units, vacuum distillation units, alkylation units, catalytic cracking units, hydrodesulfurization units, and hydrotreating units, for example. Distillate range products may have many synonyms including middle distillates or gasoil and may include hydrocarbons which boil in a range of about 180° C. to about 360° C. at 101.325 kPa. Distillate range products may include specific products such as extra light heating oil, distillate fuel oil, diesel fuel, marine diesel oil, jet fuel, and kerosene, for example. The distillate range products produced by the methods disclosed herein may have carbon numbers that are double or triple the carbon numbers of the naphtha range alkanes which the distillate range products are derived from. For example, the distillate range products may have carbon numbers ranging from C₁₂ to C₃₆ such as n-dodecane through n-hexatriacontane and isomers thereof.

The methods and systems described herein utilize oxygenate intermediates to reduce process severity and energy requirements for producing distillate range products from light alkanes. The process may include the following steps: (A) oxidation of alkanes to produce oxygenate species; (B) condensation of the oxygenate species to produce condensed species; and (C) hydro-finishing of the condensed species to produce distillate range products. In some embodiments, alkanes may be fed to an oxidation unit which may selectively oxidize the alkanes to an oxygenate product stream comprising alcohols and ketones with substantially the same carbon number as the naphtha range alkanes. Thereafter, the oxygenate product stream may be fed to a condensation unit which may condense alcohols and ketones from the oxygenate product stream to produce a condensed product stream comprising products with double or triple the carbon numbers of the naphtha range alkanes. Finally, the condensed product stream may be hydro-finished to produce a distillate range product stream.

In one aspect, the invention provides for the selective conversion of alkanes to their corresponding alcohols. In certain embodiments, the obtained alcohols are then reacted to make parrafins, alkenes, and other fuel components.

In general, the process of forming a fuel component from an alkane comprises the following steps. The conversion of n-butane is shown as an example, but the invention is not limited to conversion of n-butane:
Step 1: n-butane is converted to isobutane via isomerization over a bifunctional Pd*-acid catalyst;
Step 2: isobutane is converted to tert-butanol (TBA), acetone, and methanol via selective oxidation;
Step 3: The TBA from Step 2 is dehydrated, dimerized, and hydrogenated to make C8 paraffins; acetone is converted to iso-propanol which dehydrated to propylene; and methanol is converted to dimethyl ether which is converted to ethylene and propylene.

This reaction is described in detail in Scheme 1

### Scheme 1 Chemistry Scheme

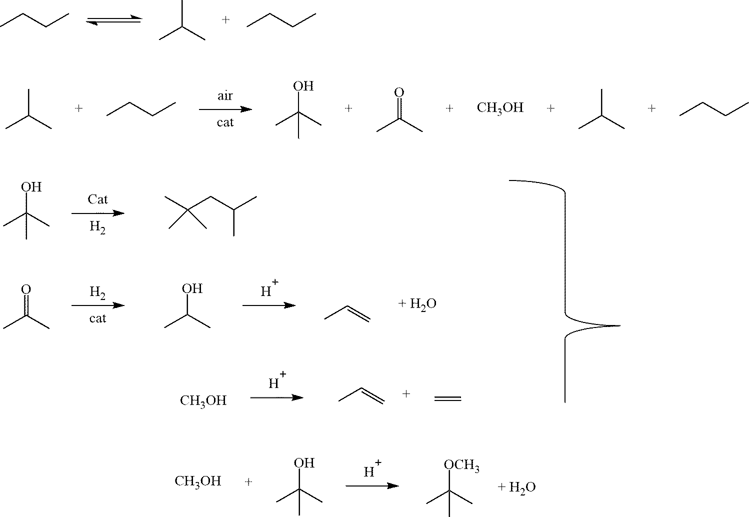

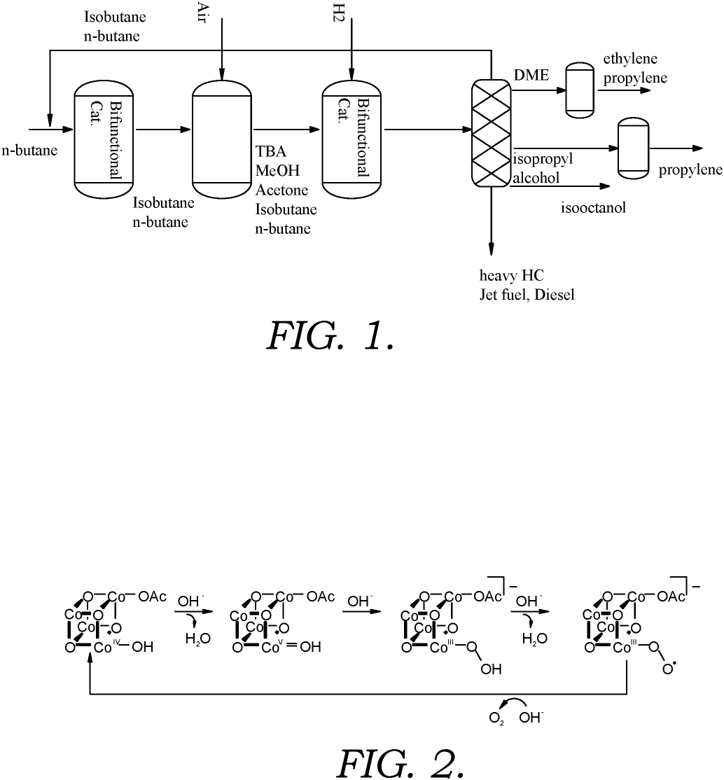

In addition, FIG. 1 provides a flowchart depicting the conversion of n-butane to isobutane using a bifunctional catalyst. This is followed by oxidation of isobutane in the presence of air to form TBA, MeOH, and acetone. These components are further reacted in the presence of hydrogen gas and an additional bifunctional catalyst to produce the products of the reaction.

In particular, methods of the description are particularly suited for the selective and efficient conversion of alkanes to the corresponding alcohol components. In particular, the claimed invention provides an oxidation step, by means of a heterogeneous catalyst, that selectively oxidizes isobutane to TBA at faster reaction rate.

The benefit of this process is the conversion of low cost n-butane or isobutane to high value liquid transportation fuels and chemicals via efficiently coupled catalytic process steps.

The TBA and by-products reactions occur in one reactor using stacked bed reactor using bifunctional catalysts which has acid and hydrogenation functionality

The oxidation of iso-butane to tert-butanol (TBA) is a reaction where two successive steps are involved (Scheme 2). The first one is the formation of tert-butyl hydroperoxide (TBHP), the second one is the decomposition of the later to provide the corresponding alcohol and by products.

### Scheme 2. Isobutane Oxidation

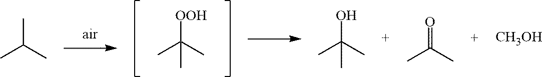

The established non-catalytic process take places at 130° C., 500 psig for 8 hours to provide the product TBA and TBHP with a conversion of 25%, with 95 % selectivity, and a molar ratio TBHP/TBA 1.2. Using the methods of the invention, the reaction rate is enhanced up to four-fold. This means a conversion around 20-25 % in 2 hours by employing a catalyst while maintaining selectivity to the TBA around 90 %.

In some embodiments, the oxidation step is carried out in the presence of one or more catalysts. In particular embodiments, the oxidation step is carried out in the presence of a cobalt cubane cluster. In other particular embodiments, the oxidation step is carried out in the presence of a cobalt cubane cluster and an additional catalyst.

Oxidation of alkanes may be carried out in liquid phase or in gaseous phase. In some examples, the alkanes may be oxidized in the liquid phase via auto-oxidation. The oxidation reaction may follow a radical reaction pathway giving secondary alcohols as the primary product with the same carbon number as the corresponding naphtha range alkane the secondary alcohol was synthesized from. The produced alcohols may be reactive and therefore prone to further oxidation which may produce ketones as well as smaller oxygenate species such as carboxylic acids and aldehydes. The resultant oxygenate product stream may comprise a mixture of unreacted naphtha range alkane, and a mixture of oxygenate species including alcohols, ketones, carboxylic acids, and aldehydes.

Any suitable source of oxygen may be used in the oxidation step. In some examples it may be desired that the oxygen-to-hydrocarbon vapor ratio may be maintained outside the explosive regime. For example, source of oxygen may include air (approximately 21 vol % oxygen), a mixture of nitrogen and oxygen, or pure oxygen. The mixture of nitrogen and oxygen may contain, for example, about 1 vol % to about 20 vol % oxygen (or greater).

The oxidation step may occur in an oxidation unit which includes equipment to facilitate the oxidation reaction. The oxidation unit may include a reactor and supporting equipment to control the oxidation reaction, add reactants, remove products, and maintain and control pressure and temperature. The oxidation step may occur at any suitable oxidation conditions, including temperature, pressure, and residence time. For example, the oxidation may occur at a temperature of about 50° C. or greater. In some embodiments, the temperature of the oxidation may range from about 50° C. to about 200° C. or, alternatively, from about 130° C. to about 160° C. In some embodiments, the oxidation reaction may be carried out at a pressure of about 500 kPa about 10100 kPa. Alternatively, the oxidation reaction may be carried out at a pressure of about 500 kPa to about 5000 kPa, about 5000 kPa to about 7500 kPa, or about 7500 kPa to about 10100 kPa. In some embodiments, the residence time in the oxidation unit may be about 0.1 hours to about 20 hours, about 0.1 hours to about 1 hour, about 1 hour to about 5 hours, or about 5 hours to about 10 hours, or about 10 to 20 hours. The oxidation reaction may be carried out in a continuous or batch process and the residence time may be selected to give a conversion to the oxygenate product of about 10% to about 40%, or greater.

In particular aspects, the oxidation methods of the invention are run at supercritical conditions.

Optionally one or more solvent(s) can be used for a process of the present disclosure. The solvent may be a polar solvent, such as benzonitrile, acetonitrile, sulfolane, carbon disulfide, nitromethane, nitrobenzene; fluorinated compounds which has hi affinity to oxygen; a saturated hydrocarbon solvent, such as n-hexane, n-heptane, cyclohexane; an aromatic solvent, such as n-hexane, n-heptane, cyclohexane, benzene, toluene, xylenes; or a mixtures thereof. In particular embodiments, the oxidation reaction is enhanced by the inclusion of e.g., benzonitrile in an amount so as to produce a mixture comprising from 10 wt % to 90 wt %, typically 20 wt % to 80 wt %, of the solvent.

The solvent mixture, the catalyst and the oxygen (e.g., oxygen-containing gas such as air) are supplied to the oxidation reaction in such proportions that the liquid phase molar ratio of solvent to dissolved oxygen is less than or equal to 20,000:1, typically less than or equal to 2,000:1, for example from 100:1 to 2000:1 and the molar ratio of solvent to cyclic imide is less than or equal to 10,000:1, typically less than or equal to 2,000:1, for example from 10:1 to 2000:1. Suitable conditions for the oxidation step include a temperature between about 70° C. and about 200° C., such as about 90° C. to about 130° C., and a pressure of about 50 kPa to 10,000 kPa. A basic buffering agent may be added to react with acidic by-products that may form during the oxidation. In addition, an aqueous phase may be introduced. The reaction can take place in a batch or continuous flow fashion.

The reactor used for the oxidation reaction may be any type of reactor that allows for introduction of oxygen to the solvent and the feedstock/ For example, the oxidation reactor may comprise a simple, largely open vessel with a distributor inlet for the oxygen-containing stream. In various embodiments, the oxidation reactor may have means to withdraw and pump a portion of its contents through a suitable cooling device and return the cooled portion to the reactor, thereby managing the heat generated in the oxidation reaction. Alternatively, cooling coils providing indirect cooling, say by cooling water, may be operated within the oxidation reactor to remove the generated heat. In other embodiments, the oxidation reactor may comprise a plurality of reactors in series, each conducting a portion of the oxidation reaction, optionally operating at different conditions selected to enhance the oxidation reaction at the pertinent conversion stages. The oxidation reactor may be operated in a batch, semi-batch, or continuous flow manner.

Any suitable technique for condensation of the oxygenate species to produce condensed products may be utilized. In embodiments, the technique selected to produce condensed products may be dependent upon the composition of the oxygenate product stream produced. For example, if the oxygenate product stream of Step (1) comprises alcohols, alcohol dehydrative dimerization or Guerbet coupling may be utilized to produce the condensed products. In alcohol dehydrative dimerization, one of the condensed products may be an olefin. In Guerbet coupling, one of the condensed products may be an alcohol, wherein the alcohol is heavier than the reactant alcohols. Alternatively, if the oxygenate product stream comprises alcohols and ketones, aldol condensation may be utilized to produce the condensed products. In aldol condensation, one of the condensed products may be a conjugated enone. In another embodiment where the oxygenate product steam comprises alcohols and ketones, selective hydrogenation of the alcohol/ketone mixture to alcohols followed by alcohol dehydrative dimerization or Guerbet coupling may be utilized to produce the condensed products. The selective hydrogenation may occur separately or in the same reactor utilized for the condensation reactions. In the embodiment where the selective hydrogenation occurs in the condensation reactor, a hydrogen co-feed with a H₂/oxygenates mole ratio of about 0.1 to about 5, about 0.1 to about 2, or about 0.1 to about 1 may be provided. A top portion of a catalyst bed disposed within the reactor may be loaded with a selective hydrogenation catalysts such as supported metal catalysts. Supported metal catalysts may be any catalyst which promotes hydrogenation such as those containing Co, Ni, Fe, Pt, Pd, Rh, Ru, Ir, Zn, Cu, Sn, Ga or combinations thereof which may be supported on silica, alumina, or titania, for example.

In Reaction (2), the dehydrative dimerization may carried out in the presence of an acid. Suitable acids may include, but are not limited to protic liquid acids such as sulfuric acid, hydrochloric acid, or sulfonic acid, solid acids such as acidic metal oxides including W/ZrO₂ and sulfated zirconia, amorphous aluminosilicates, acid clays, acidic resins, zeolites, silicoaluminophosphates, metal-organic frameworks (MOF), covalent organic frameworks (COF), zeolitic imidazolium frameworks (ZIF), and combinations thereof. Any suitable amount of acid catalyst may be used for catalyzing the dehydrative dimerization, including, an amount of about 0.001 mol to about 100 mol % of the total moles of reactants. Alternatively, about 0.01 mol % to about 5 mol %, about 5 mol % to about 20 mol %, about 20 mol % to about 50 mol %, or about 50 mol % to about 100 mol %.

The dehydrative dimerization reaction may occur in a condensation unit which includes equipment to facilitate the dehydrative dimerization reaction. The condensation unit may include a reactor and supporting equipment to control the dehydrative dimerization reaction, add reactants, remove products, and maintain and control pressure and temperature. The dehydrative dimerization reaction may occur at any suitable conditions, including temperature, pressure, and residence time. For example, the dehydrative dimerization reaction may occur at a temperature of about 50° C. or greater. In some embodiments, the temperature of the dehydrative dimerization reaction may range from about 50° C. to about 350° C. or greater.

The dehydrative dimerization reaction may occur at any suitable conditions, including temperature, pressure, and residence time in a stacked bed catalyst and co-feeding hydrogen. The top of the catalyst bed is an acid catalyst and the bottom is a hydrogenation catalyst. the acid catalyst will catalyze the dehydration/olegomerization and the hydrogenation catalyst will convert the olefins to paraffins. For example, the dehydrative dimerization/hydrogenation reactions may occur at a temperature of about 50° C. and or greater. In some embodiments, the temperature of the the dehydration/olegomerization and the hydrogenation reactions may range from about 50° C. to about 350° C. or greater. The dehydration/olegomerization and the hydrogenation reaction may be carried out at a pressure of about 500 kPa about 10100 kPa. Alternatively, the oxidation reaction may be carried out at a pressure of about 500 kPa to about 5000 kPa, about 5000 kPa to about 7500 kPa, or about 7500 kPa to about 10100 kPa.

Alternatively, the dehydrative dimerization may be carried out at a temperature from about 50° C. to about 150° C., or about 150° C. to about 250° C., or about 250° C. to about 350° C. In some embodiments, the dehydrative dimerization reaction may be carried out at a pressure of about 500 kPa about 10100 kPa. Alternatively, the dehydrative dimerization reaction may be carried out at a pressure of about 500 kPa to about 5000 kPa, about 5000 kPa to about 7500 kPa, or about 7500 kPa to about 10100 kPa. In some embodiments, the residence time in the condensation unit may be about 0.1 hours to about 30 hours. Alternatively, the residence may be about 0.1 hours to about 1 hours, about 1 hours to about 5 hours, about 5 hours to about 10 hours, or about 10 ours to about 30 hours. The dehydrative dimerization reaction may be carried out in a continuous or batch process.

Guerbet condensation may be catalyzed by metal/base bi-functional catalysts. The reaction pathways may include 1) dehydrogenation of the alcohol to aldehyde or ketone by the metal function; 2) aldehyde or ketone aldol condensation to unsaturated ketone/aldehyde catalyzed by the base; 3) rehydrogenation of the unsaturated ketone/aldehyde to alcohol by the metal function. Some examples of suitable metal/base bi-functional catalysts may include those which comprise a metal and a base. Some suitable metals may include transition metals of Group VI and above such as, without limitation, Pt—Ga, Pt—Sn, Pt—Zn, Pt—Ag, Fe, Ru, Ni, Co, Cu, and Au, and a base that includes alkali oxides, such as, without limitation, Na₂O, K₂O, Cs₂O, and alkali earth oxides such as MgO and BaO, rare-earth oxides La₂O₃, Y₂O₃, CeO₂, and combinations thereof. Additionally, the base may be carbonates or hydroxides of group 1 or 2 metals, hydroxycarbonates of group 2-13 metals such as hydrotalcite, MgₐAl_{b}(OH)_{c}(CO₃)_{d}(a, b, c, d are the mole fractions in the formula, which can be in the range of 0.1-5, such as 0.1-3, or 0.1-2). Any suitable amount of metal/base bi-functional catalysts may be used for catalyzing the Guerbet condensation, including, an amount of about 0.001 mol % to about 5 mol % of the total moles of reactants. Alternatively, about 0.01 mol % to about 1 mol %, about 1 mol % to about 2 mol %, or about 2 mol % to about 5 mol %.

Guerbet coupling may occur in a condensation unit which includes equipment to facilitate the Guerbet coupling reaction. The condensation unit may include a reactor and supporting equipment to control the Guerbet coupling reaction, add reactants, remove products, and maintain and control pressure and temperature. The Guerbet coupling reaction may occur at any suitable conditions, including temperature, pressure, and residence time. For example, the Guerbet coupling of Reaction (3) may occur at a temperature of about 50° C. or greater. In some embodiments, the temperature of the dehydrative dimerization reaction may range from about 50° C. to about 350° C. or greater. Alternatively, the Guerbet coupling reaction may be carried out at a temperature from about 50° C. to about 150° C., or about 150° C. to about 250° C., or about 250° C. to about 350° C. In some embodiments, the Guerbet coupling reaction may be carried out at a pressure of about 500 kPa about 10100 kPa. Alternatively, the Guerbet coupling reaction may be carried out at a pressure of about 500 kPa to about 5000 kPa, about 5000 kPa to about 7500 kPa, or about 7500 kPa to about 10100 kPa. In some embodiments, the residence time in the condensation unit may be about 0.1 hours to about 30 hours. Alternatively, the residence may be about 0.1 hours to about 1 hour, about 1 hour to about 5 hours, about 5 hours to about 10 hours, or about 10 hours to about 30 hours. The Guerbet coupling reaction may be carried out in a continuous or batch process.

Aldol condensation may occur in a condensation unit which includes equipment to facilitate the aldol condensation reaction. The condensation unit may include a reactor and supporting equipment to control the aldol condensation, add reactants, remove products, and maintain and control pressure and temperature. The aldol condensation may occur at any suitable conditions, including temperature, pressure, and residence time. For example, the aldol condensation of Reaction (4) may occur at a temperature of about 50° C. or greater. In some embodiments, the temperature of the aldol condensation reaction may range from about 50° C. to about 350° C. or greater. Alternatively, the aldol condensation may be carried out at a temperature from about 50° C. to about 150° C., or about 150° C. to about 250° C., or about 250° C. to about 350° C. In some embodiments, the aldol condensation reaction may be carried out at a pressure of about 500 kPa about 10100 kPa. Alternatively, the aldol condensation reaction may be carried out at a pressure of about 500 kPa to about 5000 kPa, about 5000 kPa to about 7500 kPa, or about 7500 kPa to about 10100 kPa. In some embodiments, the residence time in the condensation unit may be about 0.1 hours to about 30 hours. Alternatively, the residence may be about 0.1 hours to about 1 hour, about 1 hours to about 5 hours, about 5 hours to about 10 hours, or about 10 hours to about 30 hours. The aldol condensation reaction may be carried out in a continuous or batch process.

The aldol condensation Reaction may be catalyzed by a basic metal oxide. For example, some suitable base catalysts may include, but are not limited to, alkali oxides, hydroxides, carbonates, or bicarbonates, alkali earth oxides, hydroxides, or carbonates, rare-earth oxides, group 2 to 13 metal hydroxycarbonates such as hydrotalcites, group 2 to 13 metal carbonates, and combinations thereof. Any suitable amount of catalysts may be used for catalyzing the aldol condensation reaction, including, an amount of about 0.001 mol % to about 5 mol % of the total moles of reactants. Alternatively, about 0.01 mol % to about 1 mol %, about 1 mol % to about 2 mol %, or about 2 mol % to about 5 mol %.

Any suitable technique for hydro-finishing of the condensed products to produce distillate range products may be used. The condensed products utilized may be a product stream from a condensation unit, described in detail above. The composition of the condensed products from may depend upon the reaction route chosen to produce the condensed products. For example, in dehydrative dimerization the product may include an olefin, in aldol condensation the product may include a conjugated enone, and in Guerbet coupling the product may include an alcohol. By way of example, the hydro-finishing step may include hydro-finishing reactions such as reacting olefinic bonds, alcohols, and ketones with hydrogen, thereby reducing the concentration of olefins, ketones, and alcohols in the condensed products. The hydro-finished product is the distillate range product previously described.

The hydro-finishing reactions may occur in a hydro-finishing unit which includes equipment to facilitate the hydro-finishing reactions. The hydro-finishing reactions may include any reactions where hydrogen is added to a molecule. The hydro-finishing unit may include a reactor and supporting equipment to control the hydro-finishing reaction, add reactants, remove products, and maintain and control pressure and temperature. The hydro-finishing reactions may occur at any suitable conditions, including temperature, pressure, and residence time. For example, the hydro-finishing condensation may occur at a temperature of about 50° C. or greater. In some embodiments, the temperature of the hydro-finishing reaction may range from about 50° C. to about 350° C. or greater. Alternatively, the hydro-finishing reactions may be carried out at a temperature from about 50° C. to about 150° C., or about 150° C. to about 250° C., or about 250° C. to about 350° C. In some embodiments, the hydro-finishing reaction may be carried out at a pressure of about 500 kPa about 10100 kPa. Alternatively, the hydro-finishing reaction may be carried out at a pressure of about 500 kPa to about 5000 kPa, about 5000 kPa to about 7500 kPa, or about 7500 kPa to about 10100 kPa. In some embodiments, the residence time in the hydro-finishing unit may be about 0.1 hours to about 30 hours. Alternatively, the residence may be about 0.1 hours to about 1 hour, about 1 hour to about 5 hours, about 5 hours to about 10 hours, or about 10 hours to about 30 hours. The hydrogen to feed ratio is in the range of 1-100, such as 1-50, or 1-25. The hydro-finishing reaction may be carried out in a continuous or batch process.

The hydro-finishing reactions, for example, may be catalyzed by a hydrogenation catalyst. Some suitable hydrogenation catalysts may include, without limitation, late transition metals, such as Group VI and above, supported on alumina, silica, zirconia, titania, or carbon, for example. Example of the metal may include, without limitation, Cr, Mo, Mn, Re, Fe, Co, Ni, Pt, Pd, Ru, Rh, Ir, Au, Ag, Cu, Zn, Ga, and Sn. Any suitable amount of catalyst may be used for catalyzing the hydro-finishing reactions, including, an amount of about 0.001 mol % to about 5 mol % of the total moles of reactants. Alternatively, about 0.01 mol % to about 1 mol %, about 1 mol % to about 2 mol %, or about 2 mol % to about 5 mol %.

### Cobalt Cubane Clusters

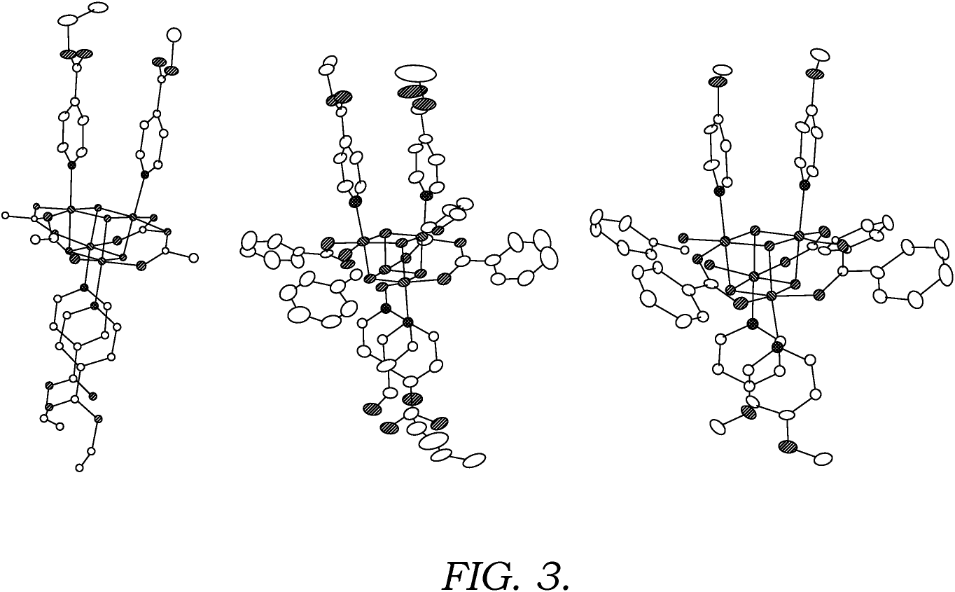

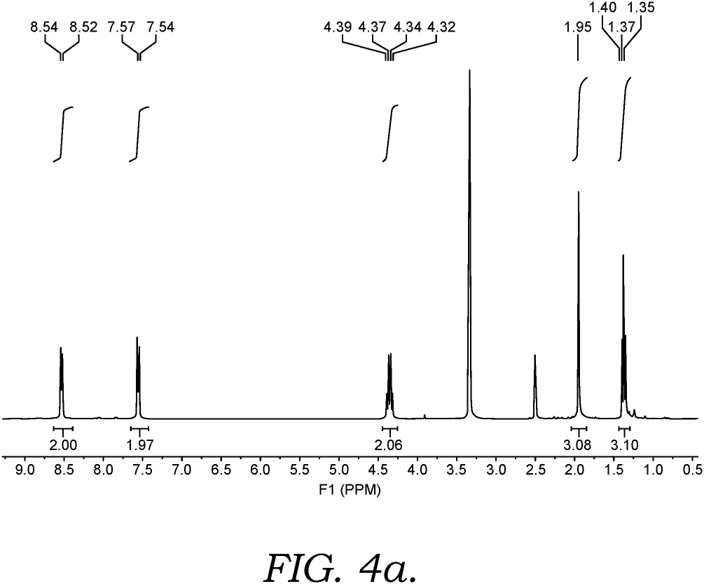

The methods of the invention utilize a cobalt catalyst based on cubane clusters. The selection of these catalysts is based on the role assigned to {Co₄O₄} clusters to form the O-O bond (See, FIG. 2) in the water splitting process (J. Am. Chem. Soc. 2015, 137, 1286), where it was elucidated that the specie B, cobalt-oxo terminal, reacts with a hydroxyl to generate the hydroperoxy-cubane C, which evolves to the radical-peroxo-cubane D before to release the O₂ molecule. On the other hand the combination of the cubane cobalt cluster catalyst which can activate oxygen and initiate the formation of peroxides with another metal which is capable of decomposing the peroxide. Without being bound by theory, it is believed that both processes are complementary, and consequently, cobalt clusters can promote hydroperoxide formation while other metals can perform the hydroperoxide decomposition.

In one aspect, the invention provides a cobalt (III) cluster with cubane structure. The cobalt cubane clusters have a general formula of:

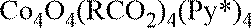

wherein R represents an aromatic or aliphatic group; and
Py* is pyridine or a pyridine functionalized at 4 position.

In certain embodiments, R is an alkyl group, an alkenyl group, an alkynyl group, benzene, pyrrole, furan, thiophene, imidazole, oxazole, thiazole, triazole, pyrazole, pyridine, pyrazine, pyridazine or pyrimidine.

Aliphatic groups include alkyl groups, alkenyl groups and alkynyl groups. In complex structures, the chains can be branched or cross-linked. Alkyl groups include saturated hydrocarbons having one or more carbon atoms, including straight-chain alkyl groups and branched-chain alkyl groups. Such hydrocarbon moieties may be substituted on one or more carbons with, for example, a halogen, a hydroxyl, a thiol, an amino, an alkoxy, an alkylcarboxy, an alkylthio, or a nitro group. Unless the number of carbons is otherwise specified, “lower aliphatic” as used herein means an aliphatic group, as defined above (e.g., lower alkyl, lower alkenyl, lower alkynyl), but having from one to six carbon atoms. Representative of such lower aliphatic groups, e.g., lower alkyl groups, are methyl, ethyl, n-propyl, isopropyl, 2-chloropropyl, n-butyl, sec-butyl, 2-aminobutyl, isobutyl, tert-butyl, 3-thiopentyl and the like. As used herein, the term “nitro” means —NO2; the term “halogen” designates -F, -Cl, -Br or -I; the term “thiol” means SH; and the term “hydroxyl” means —OH. Thus, the term “alkylamino” as used herein means an alkyl group, as defined above, having an amino group attached thereto. Suitable alkylamino groups include groups having 1 to about 12 carbon atoms, advantageously from 1 to about 6 carbon atoms. The term “alkylthio” refers to an alkyl group, as defined above, having a sulfhydryl group attached thereto. Suitable alkylthio groups include groups having 1 to about 12 carbon atoms, advantageously from 1 to about 6 carbon atoms. The term “alkylcarboxyl” as used herein means an alkyl group, as defined above, having a carboxyl group attached thereto. The term “alkoxy” as used herein means an alkyl group, as defined above, having an oxygen atom attached thereto. Representative alkoxy groups include groups having 1 to about 12 carbon atoms, advantageously 1 to about 6 carbon atoms, e.g., methoxy, ethoxy, propoxy, tert-butoxy and the like. The terms “alkenyl” and “alkynyl” refer to unsaturated aliphatic groups analogous to alkyls, but which contain at least one double or triple bond respectively. Suitable alkenyl and alkynyl groups include groups having 2 to about 12 carbon atoms, advantageously from 1 to about 6 carbon atoms.

The term “alkyl” includes saturated aliphatic groups, including straight-chain alkyl groups, branched-chain alkyl groups, cycloalkyl (alicyclic) groups, alkyl substituted cycloalkyl groups and cycloalkyl substituted alkyl groups. In certain embodiments, a straight chain or branched chain alkyl has 30 or fewer carbon atoms in its backbone, e.g., C1-C30 for straight chain or C3-C30 for branched chain. In certain embodiments, a straight chain or branched chain alkyl has 20 or fewer carbon atoms in its backbone, e.g., C1-C20 for straight chain or C3-C20 for branched chain, and more advantageously 18 or fewer. Likewise, advantageous cycloalkyls have from 4-10 carbon atoms in their ring structure and more advantageously have 4-7 carbon atoms in the ring structure. The term “lower alkyl” refers to alkyl groups having from 1 to 6 carbons in the chain and to cycloalkyls having from 3 to 6 carbons in the ring structure.

Moreover, the term “alkyl” (including “lower alkyl”) as used throughout the specification and Claims includes both “unsubstituted alkyls” and “substituted alkyls”, the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfate, sulfonato, sulfamoyl, sulfonamido, nitro, trifluoromethyl, cyano, azido, heterocyclyl, aralkyl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl, e.g., having 1 to 3 separate or fused rings and from 6 to about 18 carbon ring atoms, e.g., phenylmethyl (benzyl).

The term “amino,” as used herein, refers to an unsubstituted or substituted moiety of the formula —NRaRb, in which Ra and Rb are each independently hydrogen, alkyl, aryl, or heterocyclyl, or Ra and Rb, taken together with the nitrogen atom to which they are attached, form a cyclic moiety having from 3 to 8 atoms in the ring. Thus, the term “amino” includes cyclic amino moieties such as piperidinyl or pyrrolidinyl groups, unless otherwise stated. An “amino-substituted amino group” refers to an amino group in which at least one of Ra and Rb, is further substituted with an amino group.

The term “aromatic group” includes unsaturated cyclic hydrocarbons containing one or more rings. Aromatic groups include 5- and 6-membered single-ring groups which may include from zero to four heteroatoms, for example, benzene, pyrrole, furan, thiophene, imidazole, oxazole, thiazole, triazole, pyrazole, pyridine, pyrazine, pyridazine and pyrimidine and the like. The aromatic ring may be substituted at one or more ring positions with, for example, a halogen, a lower alkyl, a lower alkenyl, a lower alkoxy, a lower alkylthio, a lower alkylamino, a lower alkylcarboxyl, a nitro, a hydroxyl, —CF3, —CN, or the like.

The term “aryl” includes 5- and 6-membered single-ring aromatic groups that may include from zero to four heteroatoms, for example, unsubstituted or substituted benzene, pyrrole, furan, thiophene, imidazole, oxazole, thiazole, triazole, pyrazole, pyridine, pyrazine, pyridazine and pyrimidine and the like. Aryl groups also include polycyclic fused aromatic groups such as naphthyl, quinolyl, indolyl and the like. The aromatic ring can be substituted at one or more ring positions with such substituents, e.g., as described above for alkyl groups. Suitable aryl groups include unsubstituted and substituted phenyl groups. The term “aryloxy” as used herein means an aryl group, as defined above, having an oxygen atom attached thereto. The term “aralkoxy” as used herein means an aralkyl group, as defined above, having an oxygen atom attached thereto. Suitable aralkoxy groups have 1 to 3 separate or fused rings and from 6 to about 18 carbon ring atoms, e.g., O-benzyl.

Py* In certain embodiments Py* is functionalized with alkoxy, allyloxy, and acetylene functional groups. In some embodiments, Py* is an alkoxy functionalized pyridine, such as 4-methoxypyridine or 4-ethoxypyridine. In other embodiments, Py* is pyridine carboxylic acid (picolinic acid) or pyridine Methylamine (picolylamine).

Cobalt cubanes of this structure can be produced, for example, using the synthetic scheme shown in Scheme 3 below.

### Scheme 3 Synthesis of the Cubane Clusters

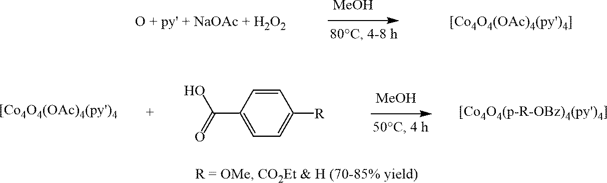

This methodology promotes cobalt clusters with high yield. The cubane clusters may also be produced using the methodology described in Dalton Trans., 2021,50, 15370-15379, which is incorporated herein by reference.

The cubane structure provides the system high stability and robustness. In addition, these catalysts are also active for CH activation under mild conditions. Moreover, these catalysts provide selectively hydroperoxide species in high yield, in order to improve the efficiency and the selectivity of the process, of isobutane oxidation into TBA and TBHP. In particular embodiments, the catalyst, when used in the process of the invention, providing selectivity of greater than 60%, more preferably greater than 75%, more preferably greater than 80%, even more preferably greater than 85% and most preferably greater than 90%. Furthermore, these catalysts provide for high yield of conversion of isobutane oxidation into TBA. In particular embodiments, the catalyst, when used in the process of the invention, provides for greater than 20% conversion, greater than 25% conversion, greater than 30% conversion, greater than 50% conversion, greater than 70% conversion, greater than 80% conversion, or greater than 90% conversion.

The cobalt cubane clusters of the invention may be used for catalyzing the oxidation reactions, including, in an amount of about 0.001 ppm to about 10 ppm of the total moles of reactants. Alternatively, about 0.01 ppm to about 7 ppm; about 0.1 ppm to about 5 ppm; or about 1 ppm to about 4 ppm.

### Additional Catalysts

The oxidation methods of the claimed invention may include an additional catalyst in combination with the cobalt cubane cluster. These catalysts include, but are not limited to, catalysts comprising palladium, ruthenium, magnesium, titanium, cerium, vanadium, manganese, nickel, zinc, tin, cobalt, silver, gold, platinum, lanthanum and compounds and mixtures thereof. In certain embodiments, the additional catalyst is a metal oxide or a mixed metal oxide. In particular embodiments, the additional catalyst is La₂O₃, CuO, MgO, CeO₂, TiO₂, V₂O₃, CoOₓ and MnOₓ.

In certain embodiments, the additional catalyst may be doped with additional metals, including, but not limited to, cobalt, gold and ruthenium. In such embodiments, the additional metal is present in a range of about 0.1 - about 4% by weight. In other such embodiments, the additional metal is present in a range of about 0.1 - about 3% by weight; of about 0.1 - about 2% by weight; or of about 0.5 - about 1% by weight.

In certain embodiments of the invention, the additional catalyst acts as a support for the cobalt cubane cluster or another metal catalyst. In particular embodiments, the additional catalyst is a Au/CeO₂ catalyst, a Ru/CeO₂ catalyst, a Ru/TiO₂ catalyst.

In particular embodiments, the additional catalyst is present as a nanoparticle. The term “nanoparticle” is a microscopic particle/grain or microscopic member of a powder/nanopowder with at least one dimension less than about 100 nm, e.g., a diameter or particle thickness of less than about 100 nm (0.1 µm), which may be crystalline or noncrystalline. Nanoparticles have properties different from, and often superior to those of conventional bulk materials including, for example, greater strength, hardness, ductility, sinterability, and greater reactivity among others. Considerable scientific study continues to be devoted to determining the properties of nanomaterials, small amounts of which have been synthesized (mainly as nano-size powders) by a number of processes including colloidal precipitation, mechanical grinding, and gas-phase nucleation and growth. Extensive reviews have documented recent developments in nano-phase materials, and are incorporated herein by reference thereto: Gleiter, H. (1989) “Nano-crystalline materials,” Prog. Mater. Sci. 33:223-315 and Siegel, R. W. (1993) “Synthesis and properties of nano-phase materials,” Mater. Sci. Eng. A168:189-197. In particular embodiments, the nanoparticles may be crystalline or amorphous. In particular embodiments, the nanoparticles may be crystalline or amorphous. In particular embodiments, the nanoparticles may form agglomerates or may be free flowing particles. In particular embodiments, the nanoparticles are less than or equal to 100 nm in diameter, e.g., less than or equal to 50 nm in diameter, e.g., less than or equal to 20 nm in diameter.

Additional catalysts may be used for catalyzing the oxidation reaction in an amount of about 0.001 mol % to about 5 mol % of the total moles of reactants. Alternatively, about 0.01 mol % to about 1 mol %, about 1 mol % to about 2 mol %, or about 2 mol % to about 5 mol %.

When used in combination with a cobalt cubane cluster of the invention, the additional catalsyts are present in an molar ratio of 1,000:1 to 1:1,000, such as from 100:1 to 1:100, such as from 50:1 to 1:50, such as from 10:1 to 1:10 with respect to the number of moles of cobalt cubane cluster.

### Listing of Embodiments

Accordingly, this disclosure provides nonlimiting embodiments as described in the following clauses.

Clause 1. A method for the production of distillate range products from light alkanes comprising:
(A) a step of oxidixing a linear or branched alkane to produce an oxygenate species;
(B) a step of condensing the oxygenate species to produce a condensed species;
and (C) a step of hydro-finishing the condensed species to produce distillate range products,
wherein the step of oxidizing a linear or branched alkane comprises reacting the alkane in the presence of one or more catalysts, wherein at least one catalyst is a cobalt cubane cluster catalyst.

Clause 2. The method according to Clause 1, wherein the alkane is a light alkane.

Clause 3. The method according to Clause 1 or 2, wherein the alkane is n-butane.

Clause 4. The method according to any one of Clauses 1 - 3, wherein the cobalt cubane cluster catalyst has the formula:

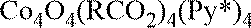

wherein R represents an aromatic or aliphatic group; and
Py* is pyridine or a pyridine functionalized at 4 position.

Clause 5. The method according to Clause 4, wherein R is methyl or phenyl.

Clause 6. The method according to any one of Clauses 4 - 5, wherein Py* is pyridine, 4-methoxy-pyridine, or 4-ethoxycarbonyl-pyridine.

Clause 7. The method according to any one of Clauses 1 - 6, wherein the cobalt cubane cluster catalyst has the formula:

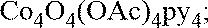

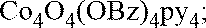

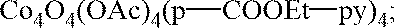

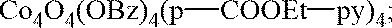

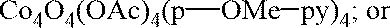

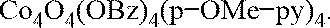

Clause 8. The method according to any one of Clauses 1 - 7, wherein the cobalt cubane cluster catalyst is present in an amount of about 0.001 ppm to about 10 ppm of the total moles of reactants.

Clause 9. The method according to any one of Clauses 1 - 8, wherein the step of oxidizing a linear or branched alkane is performed in the presence of a cobalt cubane cluster catalyst and an additional catalyst, wherein the additional catalyst is a catalyst comprising palladium, ruthenium, magnesium, titanium, cerium, vanadium, manganese, nickel, zinc, tin, cobalt, silver, gold, platinum, or lanthanum or mixtures thereof.

Clause 10. The method according to Clause 9, wherein the additional catalyst is present as a nanoparticle.

Clause 11. The method according to any one of Clauses 9 - 10, wherein the additional catalyst is present in an amount of about 0.001 mol % to about 5 mol % of the total moles of reactants.

Clause 12. The method according to any one of Clauses 9 - 11, wherein the additional catalysts are present in an molar ratio of 1,000:1 to 1:1,000 with respect to the number of moles of cobalt cubane cluster.

Clause 13. The method according to any one of Clauses 1 - 12, wherein the step of oxidizing a linear or branched alkane is performed in the presence of a solvent, wherein the solvent includes benzonitrile in an amount from 10 wt % to 90 wt % of the total solvent.

Clause 14. The method according to any one of Clauses 1 - 13, wherein the step of oxidizing a linear or branched alkane is performed under supercritical conditions.

Clause 15. The method according to any one of Clauses 1 - 14, wherein the step of oxidizing a linear or branched alkane selectively forms the alcohol component in an amount of greater than 60%.

### EXAMPLES

### Example 1. Preparation of [Co₄O₄(OAc)₄py₄] (Complex 1)

Cobalt(II) nitrate hexahydrate (10.00 g, 34.3 mmol) and sodium acetate trihydrate (9.35 g, 68.6 mmol) were dissolved in 100 mL of methanol, and then pyridine (2.8 mL, 34 mmol) was added. Hydrogen peroxide (34-37% w/w in water, 17.1 mL, 170 mmol) was added dropwise to this solution, and then the reaction mixture was refluxed for 2 h. The dark brown-green solution was dried in vacuum, and the solid was partitioned between 20 mL of water and 100 mL of dichloromethane. The organic layer was collected, and the aqueous layer was extracted with 2 × 100 mL of dichloromethane. The combined extracts were dried with MgSO₄ and concentrated to ~50 mL, to which 500 mL of hexane were added to induce crystallization. The solid was dry-loaded onto a silica column and eluted with 5% methanol in acetone. The fractions were dried to give a dark-green solid (2.01 g, 27%), which was found to be pure by ¹H NMR spectroscopy. ¹H NMR (300 MHz, DMSO-d6): δ = 8.34 (d, J = 6.5, 8H), 7.66-7.61 (t, J = 6.6 Hz, 4H), 7.15-7.11 (m, 8H), 1.92 (s, 12H). ¹³C NMR (75 MHz, DMSO-d6): δ = 183.97, 152.11, 136.92, 123.36, 26.08. Anal. Calcld for C₂₈H₃₂Co₄N₄O₁₂: C, 39.422; H, 3.754; N, 6.570; Co, 27.70. Found: C, 39.720; H, 3.978; N, 6.388; Co, 26.91.

### Example 2. Preparation of [Co₄O₄(OBz)4py₄] (Complex 2)

To prepare Co₄O₄(OBz)₄py₄ it was followed the same method as in the case of complex 1, using sodium benzoate (9.89 g, 68.6 mmol) instead of sodium acetate. For that, Co₄O₄(OAc)₄py₄ (2 g, 2.35 mmol) was dissolved in methanol and 8 equivalents of benzoic acid (2.29 g, 18.8 mmol) were added. The mixture was stirred at 50° C. for 4 hours. The solid was collected by filtration and washed with 3 × 50 mL diethyl ether. ¹H NMR (300 MHz, DMSO-d6): δ = 8.49-8.46 (d, J = 6.5 Hz, 8H), 7.81-7.77 (d, J = 7.4 Hz, 8H), 7.73-7.68 (t, J = 6.7 Hz, 4H), 7.48-7.43 (t, J = 7.4 Hz, 4H), 7.37-7.32 (m, 8H), 7.24-7.20 (m, 8H). ¹³C NMR (75 MHz, DMSO-d6): δ = 179.06, 152.10, 137.35, 135.69, 130.97, 128.29, 127.84, 123.86. Anal. Calcined for C48H40Co4N4O12: C, 52.364; H, 3.636; N, 5.091; Co, 21.43. Found: C, 52.618; H, 3.869; N, 4.685; Co, 22.13.

### Example 3. Preparation of [Co₄O₄(OAc)₄(p-COOEt-py)₄] (Complex 3)

To prepare Co₄O₄(OAc)₄(p-COOEt-py)₄ it was followed the same method as in the case of complex 1, using ethyl isonicotinate (5.14 g, 34 mmol) instead of pyridine. ¹H NMR (300 MHz, DMSO-d6): δ = 8.53 (d, J = 6.5 Hz, 8H), 7.55 (d, J = 6.6 Hz, 8H), 4.35 (q, J = 7.1 Hz, 8H), 1.95 (s, 12H), 1.37 (t, J = 7.2 Hz, 12H). ¹³C NMR (75 MHz, DMSO-d6): δ = 184.58, 163.82, 153.59, 137.41, 122.22, 61.79, 26.11, 13.94. Anal. Calcd for C40H48Co4N4O20: C, 42.100; H, 4.210; N, 4.912; Co, 20.70. Found: C, 41.720; H, 4.136; N, 4.955; Co, 20.28.

### Example 4. Preparation of [Co₄O₄(OBz)₄(p-COOEt-py)4] (Complex 4)

To prepare Co₄O₄(OBz)₄(p-COOEt-py)₄ it was followed the same method 1 as in the case of complex 1, using sodium benzoate (9.89 g, 68.6 mmol) instead of sodium acetate and ethyl isonicotinate (5.14 g, 34 mmol) instead of pyridine. For that, Co₄O₄(OAc)₄(p-COOEt-py)₄ (3.26 g, 2.35 mmol) was dissolved in methanol and 8 equivalents of benzoic acid (2.29 g, 18.8 mmol) were added. The mixture was stirred at 50° C. for 4 hours. The solid was collected by filtration and washed with 3 × 50 mL diethyl ether. ¹H NMR (300 MHz, DMSO-d6): δ = 8.70-8.68 (d, J = 6.4, 8H), 7.82-7.80 (d, J = 7.2 Hz, 8H), 7.65-7.63 (d, J = 6.5 Hz, 8H), 7.49-7.44 (t, J = 7.2 Hz, 4H), 7.37-7.32 (m, 8H), 4.36 (q, J = 7.2 Hz, 8H), 1.36 (t, J = 7.1 Hz 12H). ¹³C NMR (75 MHz, DMSO-d6): δ = 179.51, 163.78, 153.58, 137.72, 135.44, 131.14, 128.44, 127.88, 122.76, 61.81, 30.66, 13.96. Anal. Calcd for C60H56Co4N4O20: C, 51.873; H, 4.064; N, 4.034; Co, 17.00. Found: C, 52.275; H, 4.351; N, 3.619; Co, 16.89.

### Example 5. Preparation of [Co₄O₄(OAc)₄(p-OMe-py)₄] (Complex 5)

To prepare Co₄O₄(OAc)₄(p-OMe-py)₄ it was followed the same method as in the case of complex 1, using 4-methoxypyridine (3.71 g, 34 mmol) instead of pyridine. ¹H NMR (300 MHz, DMSO-d6): δ = 8.07-8.05 (d, J = 6.6 Hz, 8H), 6.76-6.74 (d, J = 6.7 Hz, 8H), 3.83 (s, 12H), 1.90 (s, 12H). ¹³C NMR (75 MHz, DMSO-d6): δ = 183.75, 165.92, 152.80, 109.88, 64.87, 26.08. Anal. Calcd for C32H40Co4N4O16: C, 39.332; H, 4.097; N, 5.736; Co, 24.14. Found: C, 38.947; H, 4.275; N, 6.006; Co, 23.35.

### Example 6. Preparation of [Co₄O₄(OBz)₄(p-OMe-py)₄] (Complex 6)

To prepare Co₄O₄(OBz)₄(p-COOEt-py)₄ it was followed the same method as in the case of complex 1, using sodium benzoate (9.89 g, 68.6 mmol) instead of sodium acetate and 4-methoxypyridine (3.71 g, 34 mmol) instead of pyridine. For that, Co₄O₄(OAc)₄(p-OMe-py)₄ (2.87 g, 2.35 mmol) was dissolved in methanol and 8 equivalents of benzoic acid (2.29 g, 18.8 mmol) were added. The mixture was stirred at 50° C. for 4 hours. The solid was collected by filtration and washed with 3 × 50 mL diethyl ether. ¹H NMR (300 MHz, DMSO-d6): δ = 8.19 (d, J = 6.4 Hz, 8H), 7.81-7.78 (m, 8H), 7.46-7.43 (t, J = 7.2 Hz, 4H), 7.37-7.32 (m, 8H), 6.85 (d, J = 7.3 Hz, 2H), 3.85 (s, 12H), 2.08 (s, 12H). ¹³C NMR (75 MHz, DMSO-d6): δ = 178.8, 167.5, 153.0, 139.9, 128.6, 128.4, 128.1 110.7, 56.0. Anal. Calcd for C52H48Co4N4O16: C, 51.146; H, 3.930; N, 4.590; Co, 19.32. Found: C, 50.627; H, 3.675; N, 4.552; Co, 19.91.

### Example 7 Structural Elucidation of the Catalysts

The structure of the developed catalysts have been fully elucidated using several techniques such as EA, ICP, NMR, CV, Raman and X-ray.

The X-ray data confirm that the proposed structures have been obtained and are stable (FIG. 1). The distances Co—O bonds are not affected by the substituent. However, the distance Co-N and the carbon from the carboxylic group are clearly elongated when the substitution in more electron donor.

Average Interatomic Bond Distances (Å) and angles (deg) for 3, 4 and 6 complexes.

TABLE 1

| | | | | | |
|---|---|---|---|---|---|
| | 3 | 4 | 6 | 1^{a} | 2^{b} |
| Co — N (py) (Å) | 1.964 | 1.959 | 1.958 | 1.962 | 1.968 |
| Co - (µ₃-O) (Å) | 1.867 | 1.864 | 1.868 | 1.865 | 1.879 |
| Co - O_{carbox} (Å) | 1.949 | 1.952 | 1.956 | 1.953 | 1.967 |
| Co ....Co^{a} (Å) | 2.835 | 2.829 | 2.824 | 2.815 | 2.856 |
| Co ....Co^{b} (Å) | 2.701 | 2.690 | 2.701 | 2.702 | 2.725 |
| O — Co — O^{c} (deg) | 84.74 | 84.88 | 85.08 | 85.21 | 84.80 |
| Co — O — Co^{c} (deg) | 94.81 | 94.66 | 94.51 | 94.97 | 94.69 |

a. Bridged by two oxo ligands only.
b. Bridged by two oxo ligands and a bidentate carboxylate.
c. Only oxygen atoms of the Co4O4 core are consider.

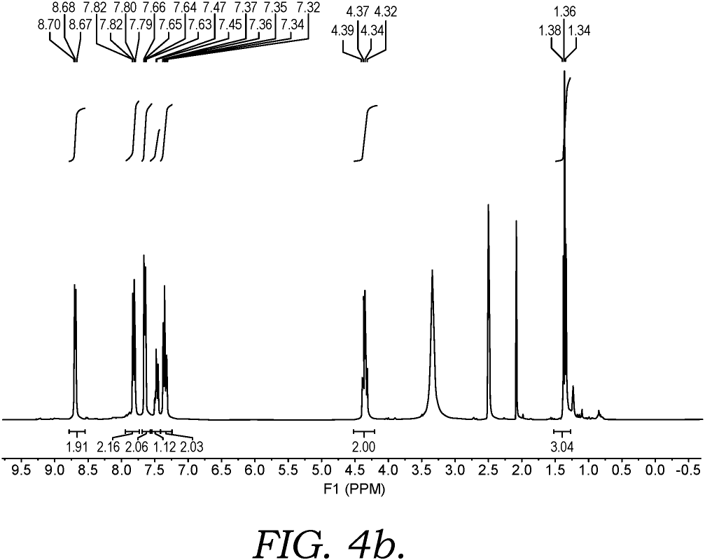

The NMR data also confirm the formation of the catalyst (FIG. 4). The FIG. 4a shows the ¹H -NMR of the complexes [Co4O4](OAc)4(py-CO2Et)4 in DMSO-d6 at room temperature. In the aliphatic region is detected the methyl group of the acetate and the ethyl groups. At 4.45 ppm is observed the quadruplet of the methylene group adjacent to the oxygen. Finally, in the aromatic region is detected the three signals of the pyridine ligands.

FIG. 4b shows the ¹H -NMR of the complexes [Co4O4](PhCO2)4(py-CO2Et)4 in DMSO-d6 at room temperature. In the aliphatic region, only the methyl of the ethyl groups is detected. At 4.45 ppm is observed the quadruplet of the methylene group adjacent to the oxygen. Finally, in the aromatic region are detected the six signals, three assigned to the pyridine ligands, and the other three to the benzoate group.

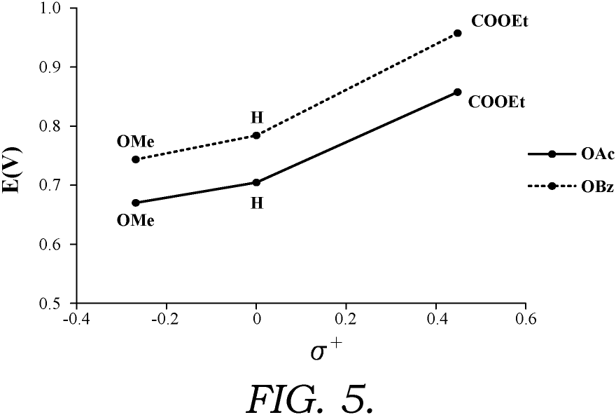

The electrochemical study based on CV experiments (FIG. 5) have provided the oxidation potential of the [Co4O4] cluster, which is modulated by the substituent on the aromatic rings. This parameter is correlated to the Lewis acidity of the metal center and therefore to the catalytic activity-selectivity (Table 1).

Table 2. Oxidation potential of cobalt clusters related to the carboxylic group (R1) and the substituent of the pyridine (R2).

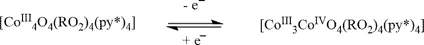

| Complex | R¹ | R² | E^{½} (V) |
|---|---|---|---|
| 1 | Me | H | 0.704 |
| 2 | Ph | H | 0.783 |
| 3 | Me | CO₂Et | 0.857 |
| 4 | Ph | CO₂Et | 0.956 |
| 5 | Me | OMe | 0.670 |
| 6 | Ph | OMe | 0.743 |

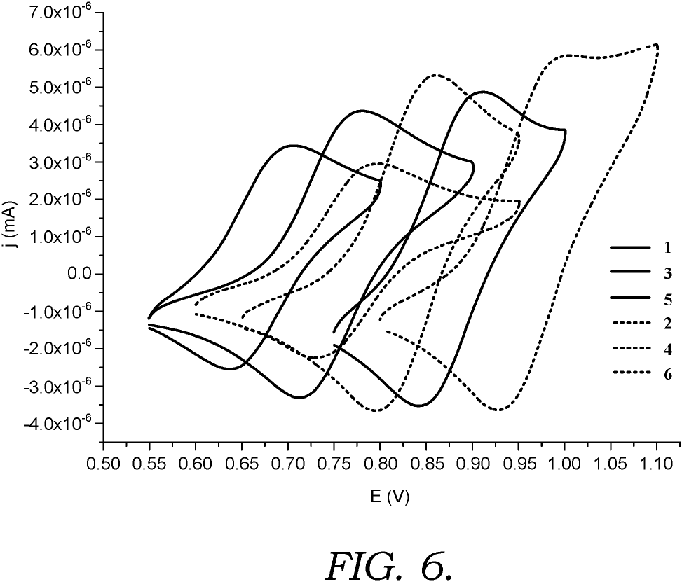

Data of oxidant potential for the clusters 1-6. (FIG. 6)These data are correlated with s⁺ values for the Hammet equation.

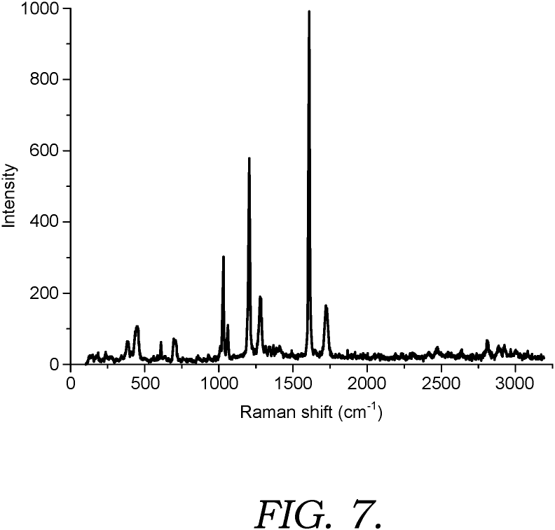

Finally, Raman spectroscopy has been also applied to characterize these complexes. The Raman spectrum of the complexes 3 is illustrated in FIG. 7.

This subtask is focused on the development of metallic nanoclusters over several heterogeneous supports based on metal oxides. On one hand, the selected supports (metal oxides) are CeO₂, TiO₂, MgO, La₂O₃, MoO and CuO. On the other, the metal clusters are based on gold, ruthenium and cobalt. Therefore, all the catalysts have been fully characterized before to evaluate their catalytic activity.

### Example 8. Gold Nanocluster Over CeO₂

The synthesis procedure of supported Au/ CeO₂ catalyst employed in this project is based in the deposition-precipitation method. The oxide (1 g) is added to 35 mL of ultrapure water and kept stirred. A second solution containing the Au precursor is prepared, 20 mg of HAuCl₄ are added to 2.43 mL of ultrapure water. Once the mixture is prepared, the pH is measured, and small amounts of NaOH 0.2 M are added until pH reaches 10. Then the solution containing the Au precursor at pH=10 is added to the CeO₂ aqueous solution. The resulting solution is kept stirred and pH is measured and kept constant at 10. Once pH is constant the solution is kept stirred overnight. Then the solid is vacuum filtrated. Finally, after filtration, it is dried in air at 100 C overnight.

Example 9. Ruthenium nanocluster over CeO₂ and TiO₂The synthesis procedure of supported Ru/CeO₂ and Ru/TiO₂ catalysts employed in this project is based impregnation and pyrolysis. A solution of the 30 mg Ru(bpy)3 in 10 mL of MeOH is added 1 g of CeO₂ or TiO₂, the mixture is stirred overnight. Afterwards, the solution is evaporated in the rotavap. Finally, the solid is introduced in an oven and heat from room temperature to 500° C. in nitrogen atmosphere. The temperature ramp is 10° C./min.

### Example 10. Cobalt Nanocluster Over CeO₂, TiO₂, MgO, La₂O₃, MoO and CuO

The synthesis of Co/MOₓ is based on the same strategy than Ru/ CeO₂ and Ru/TiO₂ catalysts. However, in this case CeO₂, TiO₂, MgO, La₂O₃, MoO and CuO are employed as supports. In addition, four types of cobalt precursors are used as Co(NO₃)₂, Co(RCO₂)₂ (where R is an alkyl group), [Co(tpy)₂]X₂ (where tpy is the abbreviature of tert-pyridine ligand and X represents an anion such as Cl, NO₃⁻ or SO₄²⁻) and the cobalt cubane clusters with structure [Co₄O₄(OAc)₄py₄].

### Example 11. Catalytic Testing

Batch reactor was used for performing the catalytic test. Isobuatane was added to the reactor as a liquid at low temperature. Then the reactor was pressurized with N2 and then with pure oxygen where the pressure was maintained by back pressure regulator. Samples were taken for GC analysis using FID (for hydrocarbon analysis and TCD for CO2, CO, H2O, H2).

### Example 12. Reactor Passivation

Before using the reactor, reactor passivation was performed with Na₄P₂O₇ to eliminate the reactor wall activity. Under this reaction conditions (Table 3a&b) rector passivation shows lower conversion and higher TBHP selectivity were obtained after passivation.

TABLE 3a

Passivate the reactor
Non-Catalytic: 130° C., DTBP, 8 hours, 6 mL of i-Butane & 30 bar N₂ & 5 bar O₂. 18% of conversion; 40% TBA, 47% TBHP, 3% acetone.

TABLE 3b

Non-Passivate the reactor
Non-Catalytic: 130° C., DTBP, 8 hours, 4 mL of i-Butane, 30 bar N₂ & 5 bar O₂. 23% of conversion; 55% TBA, 33% TBHP, 4% acetone.

Therefore, it is important that the reactor is not used previously to be passivate since metal traces do reactions fast and also product distribution.

### Example 13. Non Catalytic Iso-Butane Oxidation

Objective of this experiment is to run thermal oxidation of isobutane as a base line.

Reaction conditions 4 mL of i-Butane, 30 mg DTBP, 50 mg TBHP, 30 bar N₂ & 5 bar O₂.

Reaction temperature 130° C. data are shown herebelow

TABLE 4

thermal oxidation of isobutane as a base line

| Time | Conv | Sel(%) | | | | | | |
|---|---|---|---|---|---|---|---|---|
| (h) | % | TBOL | TBHP | acetone | DTBP | i-C4_ico | i-butanol | i-butanal |
| 1 | 5.23 | 19.26 | 43.33 | 5.37 | 31.19 | 0.00 | 0.24 | 0.60 |
| 2 | 7.49 | 22.04 | 49.82 | 4.39 | 22.96 | 0.00 | 0.18 | 0.62 |
| 3 | 10.40 | 25.41 | 53.79 | 2.41 | 17.47 | 0.00 | 0.21 | 0.70 |
| 4 | 12.58 | 27.69 | 54.72 | 2.40 | 14.34 | 0.00 | 0.19 | 0.67 |
| 8 | 18.01 | 40.77 | 46.53 | 2.87 | 9.15 | 0.00 | 0.16 | 0.53 |

The established non-catalytic process which take places at 135° C., 500 psig for 8 hours to provide the product TBA and TBHP with a conversion of 25%, with 95% selectivity, and a molar ratio TBHP/TBA 1.2.

The catalytic activity of catalyst from example 4 was evaluated, with pure oxygen and a mixture nitrogen/oxygen. These data are summarized in the table 2, where the reaction with low O₂ concentration goes faster than with pure oxygen, but the formation of acetone is also enhanced. In addition, in 2.5 hours the conversion reached 29% of conversion, being this value close to our goal 25-30% of conversion in 2 hours.

TABLE 5a

Catalytic reaction cluster 4 with pure O₂
130° C., DTBP, 4 hours, 6 mL of i-Butane & 35 bar pure O₂ with 5.8 mg of catalyst. 24% of conversion; 75% TBA, 7% TBHP, 14% acetone.

TABLE 5b

Catalytic reaction cluster 3 with N₂ & O₂
130° C., DTBP, 4 mL of i-Butane, 30 bar N₂ & 5 bar O₂ with 20 mg of catalyst.
4 hours: 35% of conversion; 72% t-BuOH, 2% TBHP, 21% acetone.
2.5 hours: 29% 72% t-BuOH, 2% TBHP, 21% acetone.

### Example 14. Impact of NHPI Highly Selective Hydrogen Donor

NHPI ( N- hydroxyl phthalimide) is well studied and found to be very active and selective promoter for free radical reactions. The presence of NHPI together with the concentration of catalysts 4 (Table 6) was evaluated, the data show that addition of NHPI decreases the selectivity to acetone dramatically

TABLE 6

100 ppm NHPI and 10 pm of 4
22% of conversion; 71% TBA, 12% TBHP, 10% acetone.

### Example 15. Impact of Temperature on Catalytic Activity of Cubane, Au/ CeO₂ and Mixed Catalyst Cubane-Au/ CeO₂

Following example 10, three reaction were performed using different catalyst
50 mg Au/ CeO₂, 4 h, 4 mL of isobutane 30 bar N2 and 5 bar O2
1 ppm of 4, 4 h, 4 mL of isobutane 30 bar N2 and 5 bar O2.
20 mg Au/ CeO₂ & 1 ppm of 4, 4 h, 4 mL of isobutane 30 bar N2 and 5 bar O2

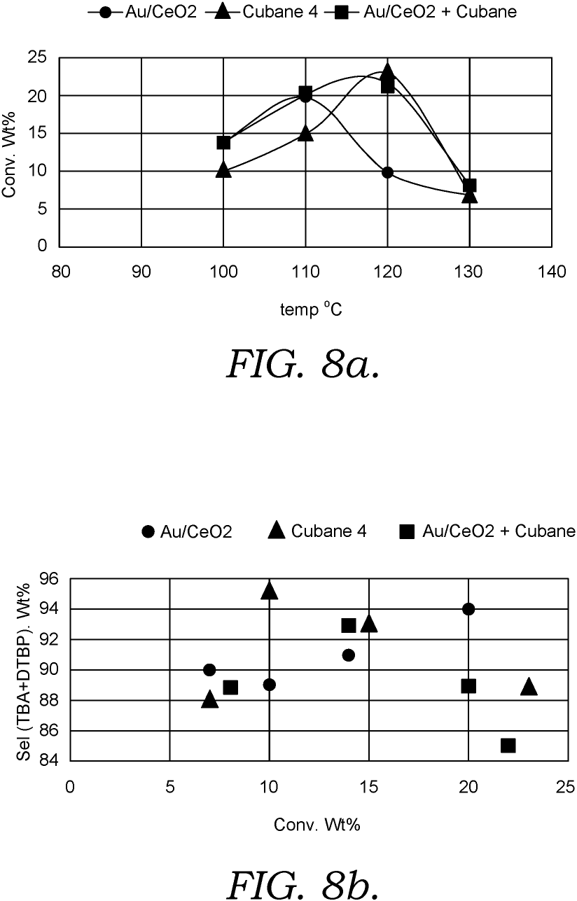

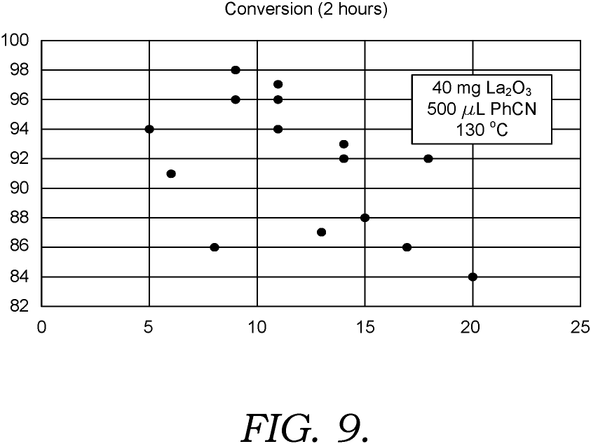

The results are shown in FIGS. 8a and 8b

The data shows that the conversion goes through a max conversion at different temperature, the same with the selectivity. Free radical chemistry is well studied and the higher the temperature the better activity is obtained. The data show that at higher temperature lower conversion is obtained. Regarding the selectivity, higher selectivity usually is obtained at lower conversion, the data show for all catalyst the selectivity goes through a max.

### Example16. Effect of Different Catalysts Combined With Cubane Catalyst

Ru/ CeO₂ was evaluated, but the conversion was low in the range from 130 to 110° C., with formation of acetone over 10%.

TABLE 7

| Entry | Temperature °C | Conversion | Selectivity | | | |
|---|---|---|---|---|---|---|
| | | | TBA | TBHP | DTBP | Acetone |
| 1 | 130 | 13 | 68 | 2 | 11 | 17 |
| 2 | 120 | 7 | 69 | 1 | 20 | 9 |
| 3 | 110 | 14 | 73 | 2 | 11 | 12 |

20 mg Ru/ CeO₂ & 1 ppm of 4, 4 h, 4 mL of isobutane 30 bar N₂ and 5 bar O₂.

The same behavior was detected when Ru/TiO₂ was employed. These data indicates that ruthenium clusters is not a proper center to carry out this process.

TABLE 8

| Entry | Temperature °C | Conversion | Selectivity | | | |
|---|---|---|---|---|---|---|
| | | | TBA | TBHP | DTBP | Acetone |
| 1 | 130 | 13 | 68 | 2 | 11 | 17 |
| 2 | 120 | 7 | 69 | 1 | 20 | 9 |
| 3 | 110 | 14 | 73 | 2 | 11 | 12 |

20 mg Ru/TiO₂ & 1 ppm of 4, 4 h, 4 mL of isobutene 30 bar N₂ and 5 bar O₂

Based on the ability of cobalt to carry out oxidation, cobalt clusters over metal oxides was evaluated. First, Co/ CeO₂ was studied, which obtained a great result at 110° C., the main problem is the ratio of acetone at 16%. In addition, 1 ppm of cobalt cubane 4 plays a role in the reaction (entry 4). Finally, catalysts loading was also evaluate (entry 5), which improves the conversion.

TABLE 9

| Entry | Temperature °C | Conversio n | Selectivity | | | |
|---|---|---|---|---|---|---|
| | | | TBA | TBHP | DTBP | Acetone |
| 1 | 130 | 13 | 66 | 1 | 17 | 14 |
| 2 | 120 | 6 | 66 | 1 | 25 | 7 |
| 3 | 110 | 24 | 77 | 1 | 5 | 16 |
| 4* | 110 | 15 | 72 | 2 | 11 | 12 |
| 5** | 110 | 27 | 76 | 1 | 6 | 17 |
| 6 | 100 | 14 | 72 | 1 | 17 | 9 |

20 mg Co/ CeO₂, 4 h, 4 mL of isobutane 30 bar N₂ and 5 bar O₂. *without 1 ppm of catalyst 4.
**40 mg of catalyst Co/ CeO₂

Moreover, the kinetic rates of the reaction with 20 mg of Co/ CeO₂ is described below. The amount of acetone goes from 11 to 16%. So, it keeps constant during the reaction.

TABLE 10

| Entry | Reaction time (h) | Conversion | Selectivity | | | |
|---|---|---|---|---|---|---|
| | | | TBA | TBHP | DTBP | Acetone |
| 1 | 1 | 10 | 77 | 1 | 9 | 11 |
| 2 | 2 | 17 | 76 | 1 | 7 | 14 |
| 3 | 3 | 21 | 77 | 1 | 5 | 15 |
| 4 | 4 | 24 | 77 | 1 | 5 | 16 |

20 mg of Co/ CeO₂, 4 h, 4 mL of isobutane 30 bar N₂ and 5 bar O₂. 1 ppm of 4.

The same behavior was observed with 40 mg of Co/ CeO₂. In this case, the amount of acetone is constant around 16%.

TABLE 11

| Entry | Reaction time (h) | Conversion | Selectivity | | | |
|---|---|---|---|---|---|---|
| | | | TBA | TBHP | DTBP | Acetone |
| 1 | 1 | 13 | 73 | 1 | 9 | 15 |
| 2 | 2 | 20 | 75 | 1 | 7 | 16 |
| 3 | 3 | 25 | 76 | 1 | 6 | 16 |
| 4 | 4 | 27 | 76 | 1 | 6 | 17 |

40 mg of Co/ CeO₂, 4 h, 4 mL of isobutane 30 bar N₂ and 5 bar O₂. 1 ppm of 4.

The main conclusions are: 1) the conversion is higher in the first two hours than in the others; 2) acetone formation is high at different reaction time; In fact, 20 % of conversion in two hours achieves the main goal of this project. However, the selectivity is not in the target

On the other hand, the cobalt cluster over TiO₂ has also shown a good activity for isobutane oxidation but with the same problem related to acetone formation.

TABLE 12

| Entry | Temperature °C | Conversion | Selectivity | | | |
|---|---|---|---|---|---|---|
| | | | TBA | TBHP | DTBP | Acetone |
| 1 | 120 | 14 | 68 | 2 | 13 | 14 |
| 2 | 110 | 21 | 77 | 5 | 5 | 11 |
| 3* | 110 | 21 | 80 | 4 | 3 | 12 |

20 mg Co/TiO₂, 4 h, 4 mL of isobutane 30 bar N₂ and 5 bar O₂. *40 mg of catalysts.

Other supports, such as ZrO₂, MgO, MoO, CuO and La₂O₃ have been employed. The most representative have been the examples with CuO and La₂O₃, which have provided great results just the support without the metal catalyst. In addition, the acetone formation is obtained in low yield, less than 5%. In addition, the employed of Co/CuO and Co/La₂O₃ have not provided better results than the corresponding supports without metal clusters. In fact, La₂O₃ affords to higher amounts of TBHP and lower acetone formation. Therefore, this is a proper catalyst to achieve this process.

TABLE 13

| Entry | Catalyst | Conversion | Selectivity | | | |
|---|---|---|---|---|---|---|
| | | | TBA | TBHP | DTBP | Acetone |
| 1 | CuO | 18 | 76 | 10 | 7 | 5 |
| 2 | Co/CuO | 19 | 75 | 8 | 8 | 8 |
| 3 | La₂O₃ | 18 | 67 | 18 | 11 | 3 |
| 4 | Co/La₂O₃ | 13 | 69 | 18 | 8 | 4 |

The effect of temperature was evaluated, where it was observed that increases the temperature promotes more conversion. In addition, increase the catalyst loading also improves the conversion up to 25%. In fact, this value implies the superior results related to conversion and selectivity.

TABLE 14

| Entry | Temperature °C | Conversion | Selectivity | | | |
|---|---|---|---|---|---|---|
| | | | TBA | TBHP | DTBP | Acetone |
| 1 | 110 | 18 | 67 | 18 | 11 | 3 |
| 2 | 130 | 21 | 63 | 21 | 8 | 7 |
| 3 | 130* | 25 | 65 | 24 | 5 | 5 |
| 4 | 130** | 14 | 54 | 31 | 11 | 4 |

20 mg of La₂O₃, 4 h, 4 mL of isobutane 30 bar N₂ and 5 bar O₂. 1 ppm of 4. *40 mg of La₂O₃. ** without 1 ppm of 4.

Then, kinetics of the reaction were analyzed, where the conversion in increase constant when the catalyst loading is 40 mg

TABLE 15

| Entry | Time (h) | Conversion | Selectivity | | | |
|---|---|---|---|---|---|---|
| | | | TBA | TBHP | DTBP | Acetone |
| 1 | 1 | 9 | 46 | 37 | 13 | 3 |
| 2 | 2 | 11 | 45 | 40 | 11 | 3 |
| 3 | 3 | 18 | 56 | 33 | 7 | 3 |
| 4 | 4 | 25 | 65 | 24 | 5 | 5 |

40 mg of La₂O₃, 130° C., 4 mL of isobutane 30 bar N₂ and 5 bar O₂. 1 ppm of 4.

### Example 17. Impact of Perfluorinated and Benzonitrile Solvents on Activity and Selectivity

Perfluorinated solvents were used in order to control the acetone formation. The selected temperature was 100° C. to promote two phases in the reactor. However, the formation of acetone is higher with perfluorinated solvent than without this solvent.

TABLE 16

| Entry | C₇F₁₆ | Conversion | Selectivity | | | |
|---|---|---|---|---|---|---|
| | | | TBA | TBHP | DTBP | Acetone |
| 1 | | 14 | 72 | 1 | 17 | 9 |
| 2 | 0.5 mL | 20 | 77 | 2 | 5 | 15 |

20 mg of Co/ CeO₂, 4 h, 100° C., 4 mL of isobutane 30 bar N₂ and 5 bar O₂. 1 ppm of 4.

In order to improve the conversion, a solvent was incorpoated. Two solvents were used - trifluorotoluene and benzonitrile. On one hand, the non-coordinated solvent, trifluorotoluene, improves the conversion with the time.

TABLE 17

| Entry | Time (h) | Conversion | Selectivity | | | |
|---|---|---|---|---|---|---|
| | | | TBA | TBHP | DTBP | Acetone |
| 1 | 1 | 7 | 64 | 19 | 9 | 7 |
| 2 | 2 | 14 | 69 | 18 | 5 | 7 |
| 3 | 3 | 21 | 73 | 15 | 3 | 8 |
| 4 | 4 | 26 | 75 | 13 | 3 | 8 |

40 mg of La₂O₃, 130° C., 4 mL of isobutane 30 bar N₂ and 5 bar O₂. 1 ppm of 4, 500 mL of triflorotoluene.

On the other hand, the coordinated solvent such as benzonitrile improves lightly the conversion. So, more coordinated solvent decrease the inhibition by products.

TABLE 18

| Entry | Time (h) | Conversion | Selectivity | | | |
|---|---|---|---|---|---|---|
| | | | TBA | TBHP | DTBP | Acetone |
| 1 | 1 | 10 | 59 | 25 | 8 | 7 |
| 2 | 2 | 18 | 65 | 21 | 4 | 8 |
| 3 | 3 | 24 | 70 | 17 | 3 | 9 |
| 4 | 4 | 29 | 72 | 15 | 3 | 9 |

40 mg of La₂O₃, 130° C., 4 mL of isobutane 30 bar N₂ and 5 bar O₂. 1 ppm of 4, 500 mL of benzonitrile.

### Example 18. Impact of Alkaline Cation on Activity and Selectivity

Moreover, alkaline cations were incorporated on the CeO₂ surface in order to modify the nature and the properties of the surface. However, the acetone formation was in the same range.

TABLE 19

| Entry | Cation and ppm | Conversion | Selectivity | | | |
|---|---|---|---|---|---|---|
| | | | TBA | TBHP | DTBP | Acetone |
| 1 | Na/300 | 17 | 72 | 1 | 17 | 9 |
| 2 | Na/2000 | 15 | 74 | 2 | 6 | 13 |
| 3 | K/300 | 18 | 77 | 2 | 7 | 12 |
| 4 | K/2000 | 21 | 77 | 2 | 6 | 14 |
| 5 | Cs/300 | 17 | 78 | 2 | 7 | 11 |
| 6 | Cs/2000 | 16 | 75 | 1 | 9 | 13 |

20 mg of Co/ CeO₂, 4 h, 100° C., 4 mL of isobutane 30 bar N₂ and 5 bar O₂. 1 ppm of 4.

The data show it depends on the conditions, catalyst and solvent the catalyst activity and selectivity can be improved.

### Example 19. Cataltyic Testing

Batch reactor was used for performing the catalytic test. Isobutane was added to the reactor as a liquid at low temperature. Then the reactor was pressurized with N₂ and then with pure oxygen where the pressure was maintained by back pressure regulator. Samples were taken for GC analysis using FID (for hydrocarbon analysis and TCD for CO₂, CO, H₂O, H₂).

### Example 20. Synthesis of Lanthanum Oxide Catalysts

La₂O₃ was obtained by mixing a solution of La(CH₃COO)₃ in anhydrous acetate acid, which was closed in a reactor upon stirring and heated at 353 K for 48 h. Afterwards, the obtained gel was dried at 393 K by slow evaporation for 72 h. Finally, it was placed in a muffle furnace, and preheated at 600° C. for 4 h, subsequently, the product was post-annealed in air atmosphere at 800° C. for 4 h. Moreover, it has been evaluated several commercial La₂O₃. The employed La₂O₃ have a BET Surface Area in the range of 100 to 15 m²/g.

### Example 21. Isobutane Oxidation in the Presence of La₂O₃ and Cobalt Cubane Catalysts

The setup described in example 19 was used for oxidation of iso-butane in the presence of catalyst.

4 ml isobutane was charged with catalyst and was run at130° C. for 2 hrs. among many catalyst were evaluated (as shown in the following chart) La₂O₃, CuO, MgO, CeO₂, TiO₂, V₂O₃, CoOₓ and MnOₓ, and also their derivates doped with 1% of cobalt, gold and ruthenium (general formula such as Co/MOₓ, Au/MOₓ and Ru/MOₓ). The following conditions were the preferred, where the results met the goals 4 ml isobutane was charged with benzonitrile as solvent (500 µL) with 40 mg of La₂O₃ and 1 ppm of cubane or cobalt (II) carboxylate at 130° C. and was run for 2 hrs.

TABLE 20

iso-butane oxidation in nthe presence of La₂O₃ and Cubane catalyst

| Entry | Time (h) | Conversion | Selectivity | | | |
|---|---|---|---|---|---|---|
| | | | TBA | TBHP | DTBP | Acetone |
| 1 | 1 | 9 | 46 | 37 | 13 | 3 |
| 2 | 2 | 11 | 45 | 40 | 11 | 3 |
| 3 | 3 | 18 | 56 | 33 | 7 | 3 |
| 4 | 4 | 25 | 65 | 24 | 5 | 5 |

40 mg of La₂O₃, 130° C., 4 mL of isobutane 30 bar N2 and 5 bar O2. 1 ppm of 4.

Then the next step was to study this catalyst under supercritical conditions. Results in table 21 show that conversion is improved while the selectivity to acetone is below 6%. In addition, DTBP concentration is decreased.

TABLE 21

iso-butane oxidation in the presence of La₂O₃ and Cubane catalyst under supercritical conditions

| Entry | Time (h) | Conversion | Selectivity | | | |
|---|---|---|---|---|---|---|
| | | | TBA | TBHP | DTBP | Acetone |
| 1 | 1 | 9 | 48 | 35 | 11 | 5 |
| 2 | 2 | 14 | 53 | 36 | 5 | 3 |
| 3 | 3 | 23 | 56 | 35 | 4 | 3 |
| 4 | 4 | 28 | 60 | 32 | 2 | 5 |

40 mg of La₂O₃, 136° C., 4 mL of isobutane 30 bar N2 and 5 bar O₂. 1 ppm of cubane.

The catalyst loading was also evaluate (see table 22), when increasing La₂O₃ loading the conversion is slightly improved together with the acetone formation to 7% at four hours, but at 2 hours the conversion achieved under this conditions is 17%, which is still below the 20-22 % targeted.

TABLE 22

iso-butane oxidation in the presence of La₂O₃ impact of catalyst and Cubane catalyst

| Entry | Time (h) | Conversion | Selectivity | | | |
|---|---|---|---|---|---|---|
| | | | TBA | TBHP | DTBP | Acetone |
| 1 | 1 | 10 | 55 | 27 | 9 | 7 |
| 2 | 2 | 17 | 58 | 30 | 5 | 7 |
| 3 | 3 | 25 | 60 | 29 | 3 | 7 |
| 4 | 4 | 29 | 63 | 27 | 2 | 7 |

80 mg of La₂O₃, 136° C., 4 mL of isobutane 30 bar N2 and 5 bar O₂. 2 ppm of Cubane catalyst.

A coordinating solvent such as benzonitrile improves slightly the conversion (Table 23). When a more coordinating and non-protic solvent as benzonitrile was used, superior results were observed.

TABLE 23

impact of protic solvent on conversion selectivity

| Entry | Time (h) | Conversion | Selectivity | | | |
|---|---|---|---|---|---|---|
| | | | TBA | TBHP | DTBP | Acetone |
| 1 | 1 | 10 | 59 | 25 | 8 | 7 |
| 2 | 2 | 18 | 65 | 21 | 4 | 8 |
| 3 | 3 | 24 | 70 | 17 | 3 | 9 |
| 4 | 4 | 29 | 72 | 15 | 3 | 9 |

40 mg of La₂O₃, 130° C., 4 mL of isobutane 30 bar N2 and 5 bar O2. 1 ppm of 4, 500 µL of benzonitrile.

The same reaction was evaluated but under supercritical conditions (see table 24). Moreover, at 2 hour 22% of conversion was obtained with a selectivity of 92%, which is above the targeted objective.

TABLE 24

impact of solvent and supercritical conditions on conversion selectivity

| Entry | Time (h) | Conversion | Selectivity | | | |
|---|---|---|---|---|---|---|
| | | | TBA | TBHP | DTBP | Acetone |
| 1 | 1 | 15 | 54 | 33 | 6 | 7 |
| 2 | 2 | 22 | 54 | 35 | 3 | 7 |
| 3 | 3 | 28 | 55 | 35 | 2 | 7 |
| 4 | 4 | 33 | 57 | 33 | 2 | 7 |

40 mg of La₂O₃, 136° C., 4 mL of isobutane 30 bar N2 and 5 bar O2. 1 ppm of 4, 500 µL of benzonitrile.

The next step was to evaluate nanopowders La₂O₃ materials with a particles size around 100 nm. First, we have carried out two experiments: one at 130° C. and the other under supercritical conditions using the nano- La₂O₃ (see table 25 and 26, respectively). Table 25 contains the results at 130° C. and no catalytic differences with to the previous La₂O₃ are observed (see Table 1). On the other hand, under supercritical conditions (Table 26) the reaction rate is higher with np- La₂O₃ respect to the one with larger crystallites. However, TBHP concentration is lower in 10 points respect to the non-np one. In addition, acetone formation is increased.

TABLE 25

Impact of particle size of the La₂O₃ on conversion selectivity

| Entry | Time (h) | Conversion | Selectivity | | | |
|---|---|---|---|---|---|---|
| | | | TBA | TBHP | DTBP | Acetone |
| 1 | 1 | 9 | 60 | 22 | 9 | 7 |
| 2 | 2 | 14 | 59 | 27 | 6 | 6 |
| 3 | 3 | 18 | 61 | 28 | 4 | 6 |
| 4 | 4 | 24 | 62 | 27 | 4 | 6 |

40 mg of np_ La₂O₃, 130° C., 4 mL of isobutane 30 bar N2 and 5 bar O2. 1 ppm of 4.

TABLE 26

act of particle size of the La₂O₃ and supercritical conditions on conversion selectivity

| Entry | Time (h) | Conversion | Selectivity | | | |
|---|---|---|---|---|---|---|
| | | | TBA | TBHP | DTBP | Acetone |
| 1 | 1 | 12 | 60 | 22 | 7 | 10 |
| 2 | 2 | 19 | 62 | 22 | 4 | 10 |
| 3 | 3 | 25 | 65 | 21 | 3 | 10 |
| 4 | 4 | 33 | 68 | 19 | 2 | 10 |

40 mg of np_ La₂O₃, 136° C., 4 mL of isobutane 30 bar N2 and 5 bar O2. 1 ppm of 4.

The same two reactions (130 and 136° C.) in the presence of benzonitrile were also evaluate (Table 27 and 28). In this case, the conversion is also improved under supercritical conditions (Table 27 and Table 28). In addition, the product distributions have the same behavior than without benzonitrile. However, at 130° C. no catalytic differences between the two La₂O₃ samples were observed (Table 27 and 28).

TABLE 27

130C in the presence of benzonitrile were also evaluate

| Entry | Time (h) | Conversion | Selectivity | | | |
|---|---|---|---|---|---|---|
| | | | TBA | TBHP | DTBP | Acetone |
| 1 | 1 | 10 | 62 | 22 | 8 | 7 |
| 2 | 2 | 20 | 65 | 21 | 5 | 8 |
| 3 | 3 | 26 | 67 | 20 | 4 | 9 |
| 4 | 4 | 30 | 69 | 19 | 3 | 9 |

40 mg of np_ La₂O₃, 130° C., 4 mL of isobutane 30 bar N2 and 5 bar O₂. 1 ppm of 4, 500 µL of benzonitrile

TABLE 28

130C in the presence of benzonitrile

| Entry | Time (h) | Conversion | Selectivity | | | |
|---|---|---|---|---|---|---|
| | | | TBA | TBHP | DTBP | Acetone |
| 1 | 1 | 14 | 60 | 22 | 7 | 10 |
| 2 | 2 | 23 | 62 | 22 | 4 | 10 |
| 3 | 3 | 29 | 65 | 21 | 3 | 10 |
| 4 | 4 | 39 | 68 | 19 | 2 | 10 |

40 mg of np_ La₂O₃, 136° C., 4 mL of isobutane 30 bar N2 and 5 bar O2. 1 ppm of 4, 500 µL of benzonitrile.

The results obtained indicate that this new np- La₂O₃ catalyst is more active than the previous one since TBHP decomposition is clearly promoted. So, optimization step will be performed in the next weeks in order to improve even further.

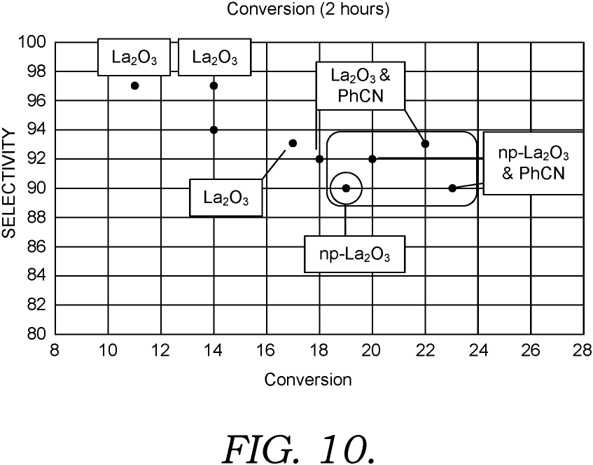

FIG. 10 illustrates conversion versus selectivity at 2 hours reaction with the described La₂O₃ and np- La₂O₃ catalysts. Now, we have three results that have achieve the main goal of this proposal.

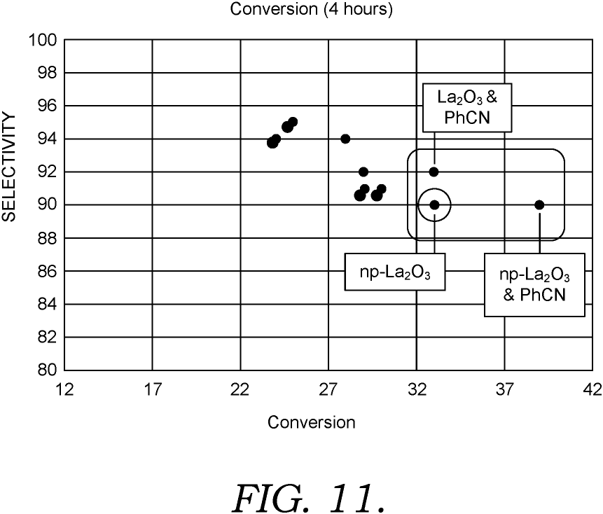

FIG. 11 represents conversion versus selectivity at 4 hours reaction with the described La₂O₃ catalysts. At 4 hours reaction, we found three results that achieve conversion higher than 33% and selectivity higher than 90%. These three results are based on La₂O₃ catalysts with 1 ppm of cubane.

### Transitional Phrases

All documents described herein are incorporated by reference herein, including any priority documents and or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a composition, an element or a group of elements is preceded with the transitional phrase “comprising,” it is understood that it also contemplates the same composition or group of elements with transitional phrases “consisting essentially of,” “consisting of,” “selected from the group of consisting of,” or “is” preceding the recitation of the composition, element, or elements and vice versa.

### Incorporation by Reference

The entire contents of all patents, published patent applications and other references cited herein are hereby expressly incorporated herein in their entireties by reference.

### Equivalents

While the present disclosure has been described with respect to a number of embodiments and examples, those skilled in the art, having benefit of this disclosure, will appreciate that other embodiments can be devised which do not depart from the scope and spirit of the present disclosure.

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired products, additional ingredients may be added, and/or similar ingredients may be substituted for one or more of the ingredients described.

Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein.

What is claimed is:

1. A method for the production of distillate range products from light alkanes comprising:
(A) a step of oxidizing a linear or branched alkane to produce at least one alcohol and one or more peroxides, a molar amount of the one or more alcohols being greater than a molar amount of the one or more peroxides;
(B) a step of condensing the at least one alcohol to produce a condensed species; and
(C) a step of hydro-finishing the condensed species to produce distillate range products,
wherein the step of oxidizing a linear or branched alkane comprises reacting the alkane in the presence of one or more catalysts, wherein at least one catalyst is a cobalt cubane cluster catalyst.

2. The method of claim 1, wherein the alkane is a light alkane.

3. The method of claim 2, wherein the alkane is n-butane.

4. The method of claim 1 wherein the cobalt cubane cluster catalyst has the formula: 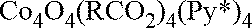
wherein R represents an aromatic or aliphatic group; and
Py* is pyridine or a pyridine functionalized at 4 position.

5. The method of claim 4, wherein R is methyl or phenyl.

6. The method of claim 4, wherein Py* is pyridine, 4-methoxy-pyridine, or 4-ethoxycarbonyl-pyridine.

7. The method of claim 4 wherein the cobalt cubane cluster catalyst has the formula: 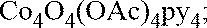 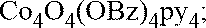 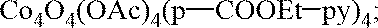 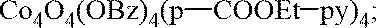 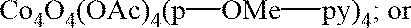 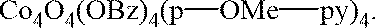
.

8. The method of claim 1, wherein the cobalt cubane cluster catalyst is present in an amount of about 0.001 ppm to about 10 ppm of the total moles of reactants.

9. The method of claim 1, wherein the step of oxidizing a linear or branched alkane is performed in the presence of the cobalt cubane cluster catalyst and an additional catalyst.

10. The method of claim 9, wherein the additional catalyst is a catalyst comprising palladium, ruthenium, magnesium, titanium, cerium, vanadium, manganese, nickel, zinc, tin, cobalt, silver, gold, platinum, or lanthanum or mixtures thereof.

11. The method of claim 10, wherein the additional catalyst is La₂O₃, CuO, MgO, CeO₂, TiO₂, V₂O₃, CoOₓ, MnOₓ, Au/CeO₂, Ru/CeO₂, or Ru/TiO₂ catalyst.

12. The method of claim 9, wherein the additional catalyst is present as a nanoparticle.

13. The method of claim 9, wherein the additional catalyst is present in an amount of about 0.001 mol % to about 5 mol % of the total moles of reactants.

14. The method of claim 9, wherein the additional catalyst is present in an molar ratio of 1,000:1 to 1:1,000 with respect to the number of moles of cobalt cubane cluster.

15. The method of claim 1, wherein the step of oxidizing a linear or branched alkane is performed in the presence of a solvent.

16. The method of claim 15, wherein the solvent includes benzonitrile in an amount from 10 wt% to 90 wt% of the total solvent.

17. The method of claim 1, wherein the step of oxidizing a linear or branched alkane is performed under supercritical conditions.

18. The method of claim 1, further comprising a step of isomerizing a linear alkane to form a branched alkane formed by isomerization, the step of oxidizing comprising oxidizing at least a portion of the branched alkane formed by isomerization.

19. A method for the production of distillate range products from light alkanes comprising:
(A) a step of oxidizing a linear or branched alkane to produc at least one alcohol and one or more peroxides;
(B) a step of condensing the at least one alcohol to produce a condensed species; and
(C) a step of hydro-finishing the condensed species to produce distillate range products,
wherein the step of oxidizing a linear or branched alkane comprises reacting the alkane in the presence of one or more catalysts, wherein at least one catalyst is a cobalt cubane cluster catalyst; and
wherein the step of oxidizing a linear or branched alkane selectively forms the at least one alcohol in an amount of greater than 60 mol%.

20. (canceled)
